(19) United States
(12) Patent Application Publication
(10) Pub. No.: US 2018/0313830 A1
(43) Pub. Date: Nov. 1, 2018

(54) PEPTIDE AND BIOMARKER ASSOCIATED WITH INFLAMMATORY DISORDERS, AND USES THEREOF

(71) Applicant: OXFORD UNIVERSITY INNOVATION LTD., OXFORD, OXFORDSHIRE (GB)

(72) Inventors: KIM MIDWOOD, OXFORD (GB); PATRICK VENABLES, OXFORD, OXFORDSHIRE (GB); ANJA SCHWENZER, OXFORD, OXFORDSHIRE (GB)

(73) Assignee: OXFORD UNIVERSITY INNOVATION LTD., OXFORD, OXFORDSHIRE (GB)

(21) Appl. No.: 15/771,244

(22) PCT Filed: Oct. 21, 2016
(86) PCT No.: PCT/GB2016/053296

§ 371 Date: Apr. 27, 2018

(30) Foreign Application Priority Data
Oct. 30, 2015 (GB) 1519175.2
Sep. 22, 2016 (GB) 1616122.6

(51) Int. Cl. G01N 33/564

(52) U.S. Cl. CPC G01N 33/564; G01N 2800/102; G01N 2333/78; G01N 2440/18

(57) ABSTRACT

The invention relates to a method of identifying a subject suspected of having or being susceptible to an autoimmune disease, such as rheumatoid arthritis (RA), comprising: contacting a sample of bodily fluid obtained from the subject with (i) a binding pair member having a binding affinity for citrullinated tenascin (cTNC) or a fragment thereof or (ii) a cTNC peptide; determining in a sample of bodily fluid obtained from the subject the presence or amount of (i) a citrullinated peptide derived from tenascin or (ii) an anti cTNC antibody; comparing the presence or amount of (i) the citrullinated peptide derived from tenascin or (ii) the anti cTNC antibody with a pre-defined threshold value; and assigning a diagnosis of RA or a future likelihood of developing RA when the presence or amount of (i) cTNC or (ii) an antibody against cTNC is detected or exceeds the threshold; and associated kits, peptides, binding members and uses thereof.

The present invention relates to citrullinated tenascin-C and its activity in chronic inflammation. In particular, the present invention relates to the use of citrullinated tenascin-C and/or autoantibodies with specificity for citrullinated tenascin-C as a biomarker for inflammatory disorders, such as rheumatoid arthritis.

Rheumatoid arthritis is a chronic disease characterized by prolonged inflammation, swelling and pain of multiple joints. With time, the chronic inflammation leads to bone destruction within the joints and to progressive disability. One prominent hallmark of rheumatoid arthritis is wide variability in its clinical presentation. This variability extends to the level of pain, number of swollen joints and extent of joint deformity. Similarly, the response of patients with rheumatoid arthritis to any specific medical therapy also varies widely, from near elimination of disease signs and symptoms in some patients, to almost complete unresponsiveness in others.

The current diagnostic approach for rheumatoid arthritis was established by the American College of Rheumatology and is composed of the following items:

1) morning stiffness lasting more than 1 hour (mainly in fingers);

2) swelling in more than 3 joints;

3) swelling in joints of hand (wrists, metacarpophalangeal joints and proximal interphalangeal joints);

4) swelling in symmetrical joints (right and left joints);

5) abnormal findings of radiography of hand;

6) subcutaneous nodules; and

7) test positive for rheumatism by a blood test for CRP (C-reactive protein) or anti-CCP (anti-cyclic citrullinated peptide). The case which satisfies more than 4 items is diagnosed as rheumatoid arthritis.

Citrullination, the conversion of arginine residues to the non-standard amino acid citrulline, is catalysed by peptidyl arginine deiminases (PAD). Levels of citrullinated proteins are significantly elevated at sites of inflammation including the joints of rheumatoid arthritis (RA) patients[1,2]. Whereas citrullination is ubiquitous in normal physiology and inflammation, antibodies to citrullinated protein antigens (ACPA) are well-established markers for the diagnosis of RA[3,4]. Appearing before evident symptoms, these autoantibodies correlate with poor prognosis and progressive joint destruction[5-8], and ACPA positive patients often require more aggressive treatment.

ACPAs are routinely detected using cyclic-citrullinated peptide (CCP) assays, designed to capture ACPA with maximum diagnostic sensitivity and specificity, using artificial peptides with no homology to naturally occurring proteins in the joint. Whilst an excellent diagnostic test, these assays are of limited use in defining subsets of ACPA positive patients and examining mechanisms of disease pathogenesis. At least 20 molecules recognised by ACPA have been described[10], but few of these have been demonstrated in the joint, epitope-mapped, antigen specificity confirmed, and evaluated in independent large cohorts. Antigenic peptides described so far that fulfil all of these criteria include citrullinated fibrinogen (cFib), citrullinated vimentin (cVim), and citrullinated α-enolase peptide 1 (CEP-1)[13]. The diagnostic sensitivity of each of these peptides is relatively low, typically between 30-50%. However, when analysed in combination, sensitivity increases, as well as demonstrating multiple serological subsets[14]. Moreover, knowledge of the antigen specificity enables investigation of aetiological mechanisms. For example gene/environment (MHC shared epitope and smoking) interactions have been demonstrated with anti-CEP-1 particularly when combined with dual positivity for anti-cVim[14]. Knowledge of the antigens involved also reveals how ACPA contribute to disease pathogenesis. For example, immune complexes containing cFib signal to induce pro-inflammatory cytokines, and antibodies to cVim provoke osteoclastogenesis and bone erosion.

Tenascin-C is a large, multimodular, extracellular matrix (ECM) glycoprotein that is specifically upregulated during inflammation, but which is absent in most healthy tissues[19,20]. Tenascin-C levels are elevated in the cartilage, synovium and synovial fluid of RA patients[21-24], as well as in RA serum where levels correlate with joint erosion[25]. Tenascin-C stimulates inflammation; inducing de novo cytokine synthesis via activation of toll-like receptor 4 (TLR4)[26], controlling cytokine synthesis post transcriptionally via induction of microRNAs and regulating adaptive immunity by driving Th17 cell polarization. In murine models of arthritis tenascin-C expression is required to maintain chronic joint inflammation and the C-terminal fibrinogen like globe domain (FBG) of tenascin-C is arthritogenic upon intra-articular injection[19].

In WO2015104563 the inventors previously identified that full length tenascin-C, as well as its individual domains, including (but not only) the FBG domain, can be post translationally modified by citrullination in vitro. The inventors showed that citrullinated FBG is better at stimulating cytokine (e.g. TNFα) synthesis by primary human macrophages than native FBG. The inventors found that only patients with a chronic inflammatory disorder, such as RA, and not normal healthy controls, possess antibodies that recognise citrullinated tenascin-C and that serum from RA patients and normal healthy controls does not react with native or non citrullinated tenascin-C. The inventors also showed that along with the FBG domain, other domains of tenascin-C are citrullinated in RA patients.

This was the first finding that tenascin-C can be citrullinated and the first demonstration that this modification of tenascin-C is relevant in RA. The inventors also showed that citrullination acts to enhance the inflammatory capacity of tenascin-C providing at least three new major mechanisms by which this protein drives inflammation in RA. The pro-inflammatory effect of the citrullinated antigen, i.e. tenascin-C, was a finding of major significance, because it shows that both antibody (e.g. via Fcγ receptor signalling) and antigen (e.g. by TLR signalling) components of ACPA-containing tenascin-C immune complexes are pro-inflammatory. Thus citrullinated tenascin-C alone, autoantibodies to citrullinated tenascin-C alone or citrullinated tenascin-C-antibody complexes may drive inflammation in disease.

However, the need remains for alternative and ever more accurate and specific markers for inflammatory conditions such as RA, particularly for detection of development of such conditions well before the onset of clinical symptoms, or at early stages of diseases when symptoms are still undifferentiated.

Therefore, an aim of the present invention is to provide alternative and more accurate and specific markers for identifying subjects afflicted with an inflammatory disorder such as RA, and particularly identifying pre-RA subjects, stratifying patients with early undifferentiated disease, or stratifying patients with diagnosed RA.

According to a first aspect of the invention there is provided a peptide comprising or consisting of the sequence R_{cit}PSNFR_{cit}NLEGR_{cit}R_{cit}, or a variant thereof, wherein R_{cit} is a citrullinated arginine residue.

The invention advantageously provides an immunodominant peptide from tenascin-C, which is distinct from the other major antigenic citrullinated peptides described to date, and is superior in terms of diagnostic sensitivity and specificity when used as an antigen in ELISA. The peptide provided the highest recorded sensitivity for any single antigenic peptide in a cohort of subjects. Furthermore, a significantly large proportion (18%) of pre-RA sera (median 7 years before diagnosis), were found to be positive for autoantibodies specific for the peptide of the invention. Thus, a significant marker for RA is provided for subjects many years before onset of the condition and allows the appropriate course of treatment or prevention to be implemented for the subject. Surprisingly citrullination of the corresponding regions of fibrinogen does not always occur at the same place as in the fibrinogen domain of tenascin C and ACPA that recognize citrullinated residues in fibrinogen do not cross react with citrullinated regions in tenascin-C.

In one aspect, a method of identifying a subject who is suspected of having or being susceptible to developing an autoimmune disease, such as rheumatoid arthritis (RA), is disclosed. The method involves the steps of (i) contacting a sample of bodily fluid obtained from the subject with (i) a binding pair member having a binding affinity for citrullinated tenascin (cTNC) or a fragment thereof or (ii) a cTNC peptide; (ii) determining in a sample of bodily fluid obtained from the subject the presence or amount of (i) a citrullinated peptide derived from tenascin or (ii) an anti cTNC antibody; (iii) comparing the presence or amount of (i) the citrullinated peptide derived from tenascin or (ii) the anti cTNC antibody with a pre-defined threshold value; and (iv) assigning a diagnosis of RA or a future likelihood of developing RA when the presence or amount of (i) cTNC or (ii) an antibody against cTNC is detected or exceeds the threshold.

The pre-defined threshold value may be at least a 90% cut off based on levels of antibody or peptide detected in individuals with no joint disease, or with a non-inflammatory joint disease such as osteoarthritis. Alternatively, the pre-defined threshold value may be at least a 92%, 95% or 98% cut off based on levels of antibody or peptide detected in individuals with no joint disease, or with a non-inflammatory joint disease such as osteoarthritis. Alternatively, the pre-defined threshold value may be at least a 99% cut off based on levels of antibody or peptide detected in individuals with no joint disease, or with a non-inflammatory joint disease such as osteoarthritis.

The autoimmune disease may comprise or consist of rheumatoid arthritis. In one embodiment, the rheumatoid arthritis is erosive rheumatoid arthritis.

The method may involve use of a reagent such as a binding pair member which has a particular binding affinity for cTNC. The binding pair member can include a monoclonal antibody, a polycloncal antibody, or functional binding fragments of each of these and may include at least one of a Fab, a Fab2, a Fv, a ScFv, a Fc, a dAb, a Fd, or a diabody. However this is a non-limiting list of fragments and other fragments having similar binding functionality might also be used.

The respective binding pair members may be purified from a mammalian host or they might be expressed using recombinant DNA technology, such as phage display, hybridoma or microbial cell culture.

In certain aspects of the method used for identifying whether a subject is suspected of having or being susceptible to developing an autoimmune disease, such as RA, the binding pair member used in the assay has specificity for cTNC in the presence of non-citrullinated TNC, such that cross reaction of the binding pair member with non-cTNC is negligible. For example, the negligible cross reaction of the binding pair member with non-cTNC may be at least 2-fold, 3-fold, 4-fold or 5-fold less cross reactivity.

In certain aspects of the method the cTNC is cTNC5 as defined in table 1.

In certain aspects of the method the cTNC has an amino acid sequence defined as RcitXXXXRcitXXXXRcitRcit, where Rcit is citrulline and X can be any amino acid. Alternatively, the cTNC can be selected from a group that includes one of the following amino acid sequences RcitXXXXRcitXXXXRcitX1; RcitXXXXRcitXXXXX1Rcit; RcitXXXXX1XXXXRcitRcit; or X1XXXXRcitXXXXRcitRcit; in which Rcit represents a citrullinated arginine residue; X represents any amino acid; and X1 represents a non-citrullinated arginine, or any other amino acid.

In a certain specific aspects, the cTNC has one of the following amino acid sequences: RcitPSNFRcitNLEGRcitRcit; EHSIQFAEMKLRcitPSNFRcitNLEGRcitRcitKR; EHSIQFAEMKLRcitPSNFRcitNLEGRcitRcitKRcit; EHSIQFAEMKLRcitPSNFRcitNLEGRcitRcitKRA; EHSIQFAEMKLRcitPSNFRcitNLEGRcitRcitKRcitA.

In certain aspects of the method the subject having or suspected of having RA is has a specific condition of erosive RA.

In one aspect of the above method the step of determining the presence or amount of a citrullinated peptide derived from tenascin can include the following:
(i) performing a sandwich immunoassay configured with a first binding pair member for cTNC associated with a solid phase and a second binding pair member with a detectable label capable of simultaneous binding to cTNC;
(ii) performing a competitive immunoassay configured with a binding pair member and labelled cTNC analog capable of competing with cTNC for binding to the binding pair member;
(iii) performing a homogeneous immunoassay comprising a binding pair member for cTNC associated with a particle, wherein the presence of cTNC results in formation of aggregates that increase turbidity of the sample;
(iv) detecting changes in the presence or amount of detectable label associated with a binding pair member or labelled cTNC analog in steps (i), (ii) or (iii); and (v) correlating changes in presence or amount of detectable label with the presence or amount of cTNC in the sample.

In another aspect of the above method the step of determining the presence or amount of an anti cTNC antibody can include the following:
(i) performing a sandwich immunoassay configured with a first binding pair member for the anti-cTNC antibody associated with a solid phase and a second binding pair member with a detectable label capable of simultaneous binding to the anti-cTNC antibody;
(ii) performing a competitive immunoassay configured with a labelled binding pair member and cTNC immobilised on a solid phase, wherein the anti-cTNC antibody competes with the binding pair member for binding to the immobilised cTNC;
(iii) performing a homogeneous immunoassay comprising a cTNC associated with a particle and a binding pair member for the anti-cTNC antibody, wherein the presence of anti-cTNC antibody results in formation of aggregates that increase the turbidity of the sample;
(iv) detecting changes in the presence or amount of detectable label associated with a binding pair member or cTNC; and
(v) correlating changes in presence or amount of detectable label with the presence or amount of anti-cTNC antibody in the sample.

In one embodiment, the peptide comprises or consists of the sequence EHSIQFAEMKLR_{cit}PSNFR_{cit}NLEGR_{cit}R_{cit}KR, or a variant peptide thereof, wherein R_{cit} is a citrullinated arginine residue. In one embodiment, the peptide comprises or consists of the sequence EHSIQFAEMKLR_{cit}PSNFR_{cit}NLEG\vec{R_{cit}}RR_{cit}KR, wherein Rcit is a citrullinated arginine residue. In another embodiment, the peptide comprises or consists of the sequence EHSIQFAEMKLR_{cit}PSNFR_{cit}NLEGR_{cit}KR_{cit}, or a variant peptide thereof, wherein \vec{R_{cit}} is a citrullinated arginine residue.

The peptide may comprise or consist of the sequence: EHSIQFAEMKLR_{cit}PSNFR_{cit}NLEGR_{cit}R_{cit}KRA, or a variant peptide thereof, wherein R_{cit} is a citrullinated arginine residue. Alternatively, the peptide may comprise or consist of the sequence: EHSIQFAEMKLR_{cit}PSNFR_{cit}NLEGR_{cit}R_{cit}KR_{cit}A, or a variant peptide thereof, wherein R_{cit} is a citrullinated arginine residue.

The peptide may comprise a variant peptide comprising or consisting of the sequence R_{cit}XXXXR_{cit}XXXXR_{cit}R_{cit}, wherein R_{cit} is a citrullinated arginine residue, and X is any amino acid.

In another embodiment, the peptide is a variant peptide comprising or consisting of any one of the sequences of

R_{cit}XXXXR_{cit}XXXXR_{cit}X₁;

R_{cit}XXXXR_{cit}XXXXX₁R_{cit};

R_{cit}XXXXX₁XXXXR_{cit}R_{cit}; or

X₁XXXXR_{cit}XXXXR_{cit}R_{cit};

wherein:

R_{cit} is a citrullinated arginine residue;

X is any amino acid; and

X₁ is a non-citrullinated arginine, or any other amino acid.

In one embodiment X₁ is a non-citrullinated arginine.

The peptide may comprise a variant peptide comprising or consisting of any one of the sequences of:

EHSIQFAEMKLR_{cit}PSNFR_{cit}NLEGR_{cit}RKR;

EHSIQFAEMKLR_{cit}PSNFR_{cit}NLEGRR_{cit}KR;

EHSIQFAEMKLR_{cit}PSNFRNLEGR_{cit}R_{cit}KR; or

EHSIQFAEMKLRPSNFR_{cit}NLEGR_{cit}R_{cit}KR; wherein R_{cit} is a citrullinated arginine residue.

In another embodiment, the peptide is a variant peptide comprising or consisting of any one of the sequences of:

EHSIQFAEMKLR_{cit}PSNFR_{cit}NLEGR_{cit}RKRA;

EHSIQFAEMKLR_{cit}PSNFR_{cit}NLEGRR_{cit}KRA;

EHSIQFAEMKLR_{cit}PSNFRNLEGR_{cit}R_{cit}KRA; or

EHSIQFAEMKLRPSNFR_{cit}NLEGR_{cit}R_{cit}KRA; wherein R_{cit} is a citrullinated arginine residue.

In another embodiment, the peptide is a variant peptide comprising or consisting of any one of the sequences of:

EHSIQFAEMKLR_{cit}PSNFR_{cit}NLEGR_{cit}RKR_{cit};

EHSIQFAEMKLR_{cit}PSNFR_{cit}NLEGRR_{cit}KR_{cit};

EHSIQFAEMKLR_{cit}PSNFRNLEGR_{cit}R_{cit}KR_{cit}; or

EHSIQFAEMKLRPSNFR_{cit}NLEGR_{cit}R_{cit}KR_{cit}; wherein R_{cit} is a citrullinated arginine residue.

In another embodiment, the peptide is a variant peptide comprising or consisting of any one of the sequences of:

EHSIQFAEMKLR_{cit}PSNFR_{cit}NLEGR_{cit}RKR_{cit}A;

EHSIQFAEMKLR_{cit}PSNFR_{cit}NLEGRR_{cit}KR_{cit}A;

EHSIQFAEMKLR_{cit}PSNFRNLEGR_{cit}R_{cit}KR_{cit}A; or

EHSIQFAEMKLRPSNFR_{cit}NLEGR_{cit}R_{cit}KR_{cit}A; wherein R_{cit} is a citrullinated arginine residue.

In one embodiment, the peptide comprises or consists of the sequence EHSIQFAEMKLR_{cit}PSNFR_{cit}NLEGRRKR, or a variant peptide thereof, wherein R_{cit} is a citrullinated arginine residue. In one embodiment, the peptide comprises or consists of the sequence EHSIQFAEMKLR_{cit}PSNFRNLEGR_{cit}RKR, or a variant peptide thereof, wherein R_{cit} is a citrullinated arginine residue. In one embodiment, the peptide comprises or consists of the sequence EHSIQFAEMKLR_{cit}PSNFRNLEGRR_{cit}KR, or a variant peptide thereof, wherein R_{cit} is a citrullinated arginine residue. In one embodiment, the peptide comprises or consists of the sequence EHSIQFAEMKLR_{cit}PSNFRNLEGRRKR_{cit}, or a variant peptide thereof, wherein R_{cit} is a citrullinated arginine residue. In one embodiment, the peptide comprises or consists of the sequence EHSIQFAEMKLRPSNFR_{cit}NLEGR_{cit}RKR, or a variant peptide thereof, wherein R_{cit} is a citrullinated arginine residue. In one embodiment, the peptide comprises or consists of the sequence EHSIQFAEMKLRPSNFR_{cit}NLEGRR_{cit}KR, or a variant peptide thereof, wherein R_{cit} is a citrullinated arginine residue. In one embodiment, the peptide comprises or consists of the sequence EHSIQFAEMKLRPSNFR_{cit}NLEGRRKR_{cit}, or a variant peptide thereof, wherein R_{cit} is a citrullinated arginine residue. In one embodiment, the peptide comprises or consists of the sequence EHSIQFAEMKLRPSNFRNLEGR_{cit}R_{cit}KR, or a variant peptide thereof, wherein R_{cit} is a citrullinated arginine residue. In one embodiment, the peptide comprises or consists of the sequence EHSIQFAEMKLRPSNFRNLEGR_{cit}RKR_{cit}, or a variant peptide thereof, wherein R_{cit} is a citrullinated arginine residue.

In one embodiment, the peptide comprises or consists of the sequence EHSIQFAEMKLR_{cit}PSNFR_{cit}NLEGRRKRA, or a variant peptide thereof, wherein R_{cit} is a citrullinated arginine residue. In one embodiment, the peptide comprises or consists of the sequence EHSIQFAEMKLR_{cit}PSNFRNLEGR_{cit}RKRA, or a variant peptide thereof, wherein R_{cit} is a citrullinated arginine residue. In one embodiment, the peptide comprises or consists of the sequence EHSIQFAEMKLR_{cit}PSNFRNLEGRR_{cit}KRA, or a variant peptide thereof, wherein R_{cit} is a citrullinated arginine residue. In one embodiment, the peptide comprises or consists of the sequence EHSIQFAEMKLR_{cit}PSNFRNLEGRRKR_{cit}A, or a variant peptide thereof, wherein R_{cit} is a citrullinated arginine residue. In one embodiment, the peptide comprises or consists of the sequence EHSIQFAEMKLRPSNFR_{cit}NLEGR_{cit}RKRA, or a variant peptide thereof, wherein R_{cit} is a citrullinated arginine residue. In one embodiment, the peptide comprises or consists of the sequence EHSIQFAEMKLRPSNFR_{cit}NLEGRR_{cit}KRA, or a variant peptide thereof, wherein R_{cit} is a citrullinated arginine residue. In one embodiment, the peptide comprises or consists of the sequence EHSIQFAEMKLRPSNFR_{cit}NLEGRRKR_{cit}A, or a variant peptide thereof, wherein R_{cit} is a citrullinated arginine residue. In one embodiment, the peptide comprises or consists of the sequence EHSIQFAEMKLRPSNFRNLEGR_{cit}R_{cit}KRA, or a variant peptide thereof, wherein R_{cit} is a citrullinated arginine residue. In one embodiment, the peptide comprises or consists of the sequence EHSIQFAEMKLRPSNFRNLEGR_{cit}RKR_{cit}A, or a variant peptide thereof, wherein R_{cit} is a citrullinated arginine residue.

The peptide may comprise a variant peptide comprising or consisting of the sequence R_{cit}XXXXR_{cit}XXXXRR, wherein R_{cit} is a citrullinated arginine residue, and X is any amino acid. The peptide may comprise a variant peptide comprising or consisting of the sequence R_{cit}XXXXRXXXXR_{cit}R, wherein R_{cit} is a citrullinated arginine residue, and X is any amino acid. The peptide may comprise a variant peptide comprising or consisting of the sequence R_{cit}XXXXRXXXXRR_{cit}, wherein R_{cit} is a citrullinated arginine residue, and X is any amino acid. The peptide may comprise a variant peptide comprising or consisting of the sequence RXXXXR_{cit}XXXXR_{cit}R_{cit}, wherein R_{cit} is a citrullinated arginine residue, and X is any amino acid. The peptide may comprise a variant peptide comprising or consisting of the sequence RXXXXRXXXXR_{cit}R_{cit}, wherein R_{cit} is a citrullinated arginine residue, and X is any amino acid.

In another embodiment, the peptide is a variant peptide comprising or consisting of any one of the sequences of

R_{cit}XXXXR_{cit}XXXXRX₁;

R_{cit}XXXXRXXXXR_{cit}X₁;

RXXXXR_{cit}XXXXR_{cit}X₁;

R_{cit}XXXXR_{cit}XXXXX₁R;

R_{cit}XXXXRXXXXX₁R_{cit};

RXXXXR_{cit}XXXXX₁R_{cit};

R_{cit}XXXXX₁XXXXR_{cit}R;

R_{cit}XXXXX₁XXXXRR_{cit};

RXXXXX₁XXXXR_{cit}R_{cit};

X₁XXXXR_{cit}XXXXR_{cit}R;

X₁XXXXR_{cit}XXXXRR_{cit};

X₁XXXXRXXXXR_{cit}R_{cit}; or

wherein:

R_{cit} is a citrullinated arginine residue;

X is any amino acid; and

X₁ is a non-citrullinated arginine, or any other amino acid.

In one embodiment X₁ is a non-citrullinated arginine.

The peptide may comprise a variant peptide comprising or consisting of any one of the sequences of:

EHSIQFAEMKLR_{cit}PSNFR_{cit}NLEGRRKR;

EHSIQFAEMKLR_{cit}PSNFRNLEGRR_{cit}KR;

EHSIQFAEMKLRPSNFRNLEGR_{cit}R_{cit}KR;

EHSIQFAEMKLRPSNFR_{cit}NLEGRR_{cit}KR; or

EHSIQFAEMKLRPSNFR_{cit}NLEGR_{cit}RKR; wherein R_{cit} is a citrullinated arginine residue.

In another embodiment, the peptide is a variant peptide comprising or consisting of any one of the sequences of:

EHSIQFAEMKLR_{cit}PSNFR_{cit}NLEGRRKRA;

EHSIQFAEMKLR_{cit}PSNFRNLEGRR_{cit}KRA;

EHSIQFAEMKLRPSNFRNLEGR_{cit}R_{cit}KRA;

EHSIQFAEMKLRPSNFR_{cit}NLEGRR_{cit}KRA; or

EHSIQFAEMKLRPSNFR_{cit}NLEGR_{cit}RKRA; wherein R_{cit} is a citrullinated arginine residue.

In another embodiment, the peptide is a variant peptide comprising or consisting of any one of the sequences of:

EHSIQFAEMKLRPSNFRNLEGR_{cit}RKR_{cit};

EHSIQFAEMKLR_{cit}PSNFRNLEGRRKR_{cit}; or

EHSIQFAEMKLRPSNFRNLEGRR_{cit}KR_{cit}; wherein R_{cit} is a citrullinated arginine residue.

In another embodiment, the peptide is a variant peptide comprising or consisting of any one of the sequences of:

EHSIQFAEMKLRPSNFRNLEGR_{cit}RKR_{cit}A;

EHSIQFAEMKLR_{cit}PSNFRNLEGRRKR_{cit}A; or

EHSIQFAEMKLRPSNFRNLEGRR_{cit}KR_{cit}A; wherein R_{cit} is a citrullinated arginine residue.

One or more non-citrullinated amino acid residues of a variant peptide according to the invention herein may be removed or added, such that the spacing between the citrullinated arginine residues is varied. In one embodiment, one or more non-citrullinated amino acid residues of a variant peptide according to the invention herein may be removed, such that the spacing between one or more pairs of citrullinated arginine residues is reduced. Alternatively, one or more non-citrullinated amino acid residues of a variant peptide according to the invention herein may be added, such that the spacing between one or more pairs of citrullinated arginine residues is increased.

The peptide may comprise a N-terminal cysteine and a C-terminal cysteine. The peptide may be in a looped or cyclic formation, for example to aid antibody recognition. In one embodiment, the peptide(s) may be affinity tagged, for example by biotin. The skilled person will understand that affinity tagging the peptide(s) according to the invention will aid there use in assay chips and ELISAs.

The skilled person will understand that one or more non-citrullinated amino acid residues of the peptide may be modified without substantially affecting the peptide function. For example, the modification may comprise addition or deletion of chemical moeities, such as charged groups without affecting function (i.e. an autoanibody may still bind). The skilled person will understand that one or more amino acid may be substituted with amino acid analogues or derivatives without affecting function.

In one embodiment, the peptide is cTNC5 peptide described herein.

According to another aspect of the present invention, there is provided a biomarker for determining the inflammatory disorder status, such as RA status, of a subject wherein the biomarker comprises:
(i) citrullinated tenascin-C or a fragment thereof which is citrullinated at at least two arginine residues of residue numbers 2187, 2192, 2197 and 2198; and/or
(ii) autoantibodies with specificity for an epitope of citrullinated tenascin-C or a fragment thereof wherein the epitope comprises at least two citrullinated arginine residues of 2187, 2192, 2197 and 2198.

In one embodiment, the biomarker may comprise citrullinated tenascin-C or a fragment thereof which is citrullinated at at least three arginine residues of residue numbers 2187, 2192, 2197 and 2198.

In one embodiment, the biomarker may comprise autoantibodies with specificity for an epitope of citrullinated tenascin-C or a fragment thereof wherein the epitope comprises at least three citrullinated arginine residues of 2187, 2192, 2197 and 2198.

In one embodiment, the citrullinated tenascin-C or a fragment thereof is citrullinated at at least two arginine residues of residue numbers 2187, 2192, 2197, 2198 and 2200. In one embodiment, the epitope comprises at least two citrullinated arginine residues of 2187, 2192, 2197, 2198 and 2200. In one embodiment, the citrullinated tenascin-C or a fragment thereof is citrullinated at at least three arginine residues of residue numbers 2187, 2192, 2197, 2198 and 2200. In one embodiment, the epitope comprises at least three citrullinated arginine residues of 2187, 2192, 2197, 2198 and 2200.

In one embodiment, the biomarker comprises citrullinated tenascin-C or a fragment thereof which is citrullinated at arginine residues 2187, 2192, 2197 and 2198. In one embodiment, the biomarker comprises citrullinated tenascin-C or a fragment thereof which is citrullinated at arginine residues 2187, 2192, 2197, 2198 and 2200. In one embodiment, the biomarker comprises autoantibodies with specificity for an epitope of citrullinated tenascin-C or a fragment thereof wherein the epitope comprises citrullinated arginine residues 2187, 2192, 2197 and 2198. In one embodiment, the biomarker comprises autoantibodies with specificity for an epitope of citrullinated tenascin-C or a fragment thereof wherein the epitope comprises citrullinated arginine residues 2187, 2192, 2197, 2198, and 2200. In one embodiment, the biomarker comprises citrullinated tenascin-C or a fragment thereof which is citrullinated at arginine residues 2187, 2192, 2197 and 2198; and autoantibodies with specificity for an epitope of citrullinated tenascin-C or a fragment thereof wherein the epitope comprises citrullinated arginine residues 2187, 2192, 2197 and 2198. In one embodiment, the biomarker comprises citrullinated tenascin-C or a fragment thereof which is citrullinated at arginine residues 2187, 2192, 2197, 2198, and 2200; and autoantibodies with specificity for an epitope of citrullinated tenascin-C or a fragment thereof wherein the epitope comprises citrullinated arginine residues 2187, 2192, 2197, 2198, and 2200.

According to another aspect of the invention, there is provided a method of determining the inflammatory disorder status of a subject comprising detecting the presence or absence, or the level, of a biomarker in a sample from said subject, wherein the biomarker comprises:
(i) citrullinated tenascin-C or a fragment thereof which is citrullinated at at least two arginine residues of residue numbers 2187, 2192, 2197 and 2198; and/or
(ii) autoantibodies with specificity for an epitope of citrullinated tenascin-C or a fragment thereof wherein the epitope comprises at least two citrullinated arginine residues of 2187, 2192, 2197 and 2198.

In one embodiment, the biomarker may comprise citrullinated tenascin-C or a fragment thereof which is citrullinated at at least three arginine residues of residue numbers 2187, 2192, 2197 and 2198.

In one embodiment, the biomarker may comprise autoantibodies with specificity for an epitope of citrullinated tenascin-C or a fragment thereof wherein the epitope comprises at least three citrullinated arginine residues of 2187, 2192, 2197 and 2198.

Residue numbers may be determined from TNC sequence Uniprot Accession No. P24821 (SEQ ID NO: 1).

In one embodiment, the method comprises detecting the presence or absence, or the level, of citrullinated tenascin-C or a fragment thereof which is citrullinated at arginine residues 2187, 2192, 2197 and 2198. In one embodiment, the method comprises detecting the presence or absence, or the level, of autoantibodies with specificity for an epitope of citrullinated tenascin-C or a fragment thereof wherein the epitope comprises citrullinated arginine residues 2187, 2192, 2197 and 2198. In one embodiment, the method comprises detecting the presence or absence, or the level, of citrullinated tenascin-C or a fragment thereof which is citrullinated at arginine residues 2187, 2192, 2197 and 2198; and detecting the presence or absence, or the level, of autoantibodies with specificity for an epitope of citrullinated tenascin-C or a fragment thereof wherein the epitope comprises citrullinated arginine residues 2187, 2192, 2197 and 2198.

In one embodiment, the citrullinated tenascin-C or a fragment thereof is citrullinated at at least two arginine residues of residue numbers 2187, 2192, 2197, 2198 and 2200. In one embodiment, the epitope comprises at least two citrullinated arginine residues of 2187, 2192, 2197, 2198 and 2200. In one embodiment, the citrullinated tenascin-C or a fragment thereof is citrullinated at at least three arginine residues of residue numbers 2187, 2192, 2197, 2198 and 2200. In one embodiment, the epitope comprises at least three citrullinated arginine residues of 2187, 2192, 2197, 2198 and 2200.

In one embodiment, the method comprises detecting the presence or absence, or the level, of citrullinated tenascin-C or a fragment thereof which is citrullinated at arginine residues 2187, 2192, 2197, 2198 and 2200. In one embodiment, the method comprises detecting the presence or absence, or the level, of autoantibodies with specificity for an epitope of citrullinated tenascin-C or a fragment thereof wherein the epitope comprises citrullinated arginine residues 2187, 2192, 2197, 2198 and 2200. In one embodiment, the method comprises detecting the presence or absence, or the level, of citrullinated tenascin-C or a fragment thereof which is citrullinated at arginine residues 2187, 2192, 2197, 2198 and 2200; and detecting the presence or absence, or the level, of autoantibodies with specificity for an epitope of citrullinated tenascin-C or a fragment thereof wherein the epitope comprises citrullinated arginine residues 2187, 2192, 2197, 2198 and 2200.

The epitope may be on a fragment of cTNC. The fragment of cTNC may comprise a sequence of the peptide according to the invention herein.

The method may comprise detecting the presence or absence, or the level, of autoantibodies with specificity for cTNC5 described herein. The method may comprise detecting the level of autoantibodies with specificity for cTNC5 described herein.

In one embodiment, the presence/detection of the biomarker in a sample from said subject is sufficient to conclude the subject has an inflammatory disorder.

The inflammatory disorder may be associated with any condition associated with inappropriate inflammation. Such disorders include, but are not limited to, rheumatoid arthritis (RA), autoimmune conditions, inflammatory bowel diseases (including Crohn's disease and ulcerative colitis), non-healing wounds, multiple sclerosis, cancer, atherosclerosis, sjogrens disease, diabetes, lupus erythrematosus (including systemic lupus erythrematosus), asthma, fibrotic diseases (including liver cirrhosis), pulmonary fibrosis, UV damage, psoriasis, psoriatic arthritis, ankylosing spondylitis, myositis and cardiovascular disease.

Of particular, but non-exclusive interest, the invention is concerned with chronic inflammation associated with rheumatoid arthritis (RA). Therefore, in one embodiment, the inflammatory condition is rheumatoid arthritis (RA).

The phrase “inflammatory disorder status” includes any distinguishable manifestation of an inflammatory disorder, and includes, without limitation, the presence or absence of an inflammatory disorder, the risk of developing an inflammatory disorder, the stage of an inflammatory disorder, the progression of an inflammatory disorder, and the effectiveness or response of a subject to a treatment for an inflammatory disorder.

In a preferred embodiment, the inflammatory disorder referred to is rheumatoid arthritis, and the method of the invention allows, without limitation, the determination of the presence or absence of rheumatoid arthritis, the risk of developing rheumatoid arthritis, the stage of rheumatoid arthritis, the progression of rheumatoid arthritis, the remission of arthritis, the best likely treatment for rheumatoid arthritis and the effectiveness or response of a subject to a treatment for rheumatoid arthritis. In one embodiment, the inflammatory disorder may be a persistent inflammatory disorder, such as RA.

The method of the invention may be used, for example, for any one or more of the following: to diagnose rheumatoid arthritis in a subject; to assess the chance of a subject developing rheumatoid arthritis; to advise on the prognosis for a subject with rheumatoid arthritis; to monitor disease progression; to advise on treatment options; to determine the likelihood of rheumatoid arthritis; to determine the likelihood of resolvable synovial inflammation; and to monitor effectiveness or response of a subject to a treatment for rheumatoid arthritis.

The method may allow a diagnosis of rheumatoid arthritis to be given in a subject with no other symptoms of rheumatoid arthritis.

The presence, and optionally the level, of the biomarker in a sample may be determined by any suitable assay, which may comprise the use of any of the group comprising immunoassays, spectrometry, western blot, ELISA, immunoprecipitation, slot or dot blot assay, isoelectric focussing, SDS-PAGE and antibody microarray immunohistological staining, radio immuno assay (RIA), fluoroimmunoassay, an immunoassay using an avidin-biotin or streptoavidin-biotin system, etc or combinations thereof. These methods are well known to persons skilled in the art. Such assay methods may include the use of microarray chips such as Phadia's ImmunoCAP ISAC system described in Hansson et al. (Arthritis Research & Therapy 2012, 14:R201), which is herein incorporated by reference.

The peptide, or multiple copies thereof, may be detected alone using such assay methods. Alternatively, the peptide of the invention, or biomarkers of the invention may be detected in combination with other known ACPA or peptides, or other citrullinated proteins. The peptide of the invention, or biomarkers of the invention may be detected in combination with other known biomarkers for inflammatory disease, such as biomarkers for RA. One example of another known biomarker is Rheumatoid Factor (RF). Another example biomarker for use in combination with the invention is CCP.

The presence, and optionally the level, of the biomarker in a sample may be determined by using an antibody specific to the biomarker that does not bind native tenascin-C, and does not bind alternative citrullinated tenascin-C, or fragments thereof, which have a different citrullination pattern. The method may further comprise immunoassaying using said antibody e.g. by ELISA and/or western blot. For either serum or tissue/cells this is preferably a sandwich ELISA, i.e. one citrullinated tenascin-C(citTNC) Ab used as capture, then adding serum/tissue lysate as suitable dilution, then a second, different citTNC antibody to detect. A second antibody can be labelled for detection or indirect detection—as in any standard ELISA protocol. In the event that two different suitable citTNC antibodies cannot be found, the method uses a direct ELISA made up of serum/tissue lysate on plates and the antibody added directly to this. Alternatively, a non-citrullinated-TNC antibody may be used for capture, and a citrullinated-TNC antibody may be used for detection, or vice versa. In another embodiment, a citrulline specific antibody may be used for detection, for example in an ELISA.

The peptide of the invention or citrullinated-TNC antibody (or antibodies thereto) may be immobilised on a solid support. An array of the peptide of the invention or citrullinated-TNC antibody (or antibodies thereto) may be immobilised on a solid support.

In one embodiment, the ratio of native TNC and the biomarker may be determined. In another embodiment, the ratio of the biomarker relative to citrullinated TNC having an alternative citrullination pattern may be determined.

Alternatively, tenascin-C and/or fragments of tenascin-C may be immune precipitated and then western blotting or mass spectrometry may be used to determine if the tenascin-C is citrullinated at the specified residues of the biomarker.

Samples containing autoantibodies having specificity for the biomarker may be determined using western blotting with RA serum as in the examples.

Alternatively, the residues that are citrullinated in tenascin-C and/or fragments of citrullinated tenascin-C may be better defined and then a peptide assay may be created (either a western blot as above, and/or an ELISA approach). Only the peptide which is citrullinated is created and, as a control, a non-citrullinated peptide. The plate is coated with peptide and RA serum applied and used to detect antibody (see Lundberg (2008) for details of the methods). The citrullinated tenascin-C peptide for use in the assay may comprise any tenascin-C peptide comprising two, three or four citrullinated residues selected from any of the group comprising residues 2187, 2192, 2197 and 2198; or combinations thereof. In one embodiment, the peptide may comprise citrullinated residues 2187, 2192, 2197 and 2198. The citrullinated tenascin-C peptide for use in the assay may comprise any tenascin-C peptide comprising two, three or four citrullinated residues selected from any of the group comprising residues 2187, 2192, 2197, 2198 and 2200; or combinations thereof. In one embodiment, the peptide may comprise citrullinated residues 2187, 2192, 2197, 2198 and 2200.

An equivalent non-citrullinated peptide of the same sequence as the biomarker may be used as the assay control.

The biomarker citrullinated tenascin-C peptide, or fragment thereof, for use in a peptide assay may be in a looped or cyclic formation, for example to aid antibody recognition.

The presence of the biomarker in a sample from said subject may be sufficient to conclude the subject has an inflammatory disorder or is at risk of developing the inflammatory disorder. The presence of the biomarker in a sample from said subject may be sufficient to conclude the subject has an inflammatory disorder or is at risk of developing the inflammatory disorder up to 16 years prior to the development of the disorder. The subject may be at risk of developing the inflammatory disorder within 25 years. The subject may be at risk of developing the inflammatory disorder within 20 years. The subject may be at risk of developing the inflammatory disorder within 19 years. The subject may be at risk of developing the inflammatory disorder within 18 years. The subject may be at risk of developing the inflammatory disorder within 17 years. The subject may be at risk of developing the inflammatory disorder within 16 years. The subject may be at risk of developing the inflammatory disorder within 15 years. The subject may be at risk of developing the inflammatory disorder within 14 years. The subject may be at risk of developing the inflammatory disorder within 13 years. The subject may be at risk of developing the inflammatory disorder within 12 years. The subject may be at risk of developing the inflammatory disorder within 11 years. The subject may be at risk of developing the inflammatory disorder within 10 years. The subject may be at risk of developing the inflammatory disorder within 9 years. The subject may be at risk of developing the inflammatory disorder within 8 years. The subject may be at risk of developing the inflammatory disorder within 7 years. The subject may be at risk of developing the inflammatory disorder within 6 years. The subject may be at risk of developing the inflammatory disorder within 5 years. The subject may be at risk of developing the inflammatory disorder within 4 years. The subject may be at risk of developing the inflammatory disorder within 3 years. The subject may be at risk of developing the inflammatory disorder within 2 years. The subject may be at risk of developing the inflammatory disorder within 1 year.

The method of the invention may comprise the further step of comparing the level of the biomarker determined in the sample with one or more reference values.

Preferably the reference value, to which the determined levels of biomarker are compared, is the level of the biomarker observed in one or more subjects that do not have any detectable inflammatory disorder, such as rheumatoid arthritis, or any clinical symptoms of an inflammatory disorder, such as rheumatoid arthritis, and have so called “normal values” of the biomarker. The “normal values” of the biomarker may be zero, or at least undetectable using assay methods described herein.

In a further embodiment the ratio of the biomarker to native tenascin-C may be considered and compared.

Alternatively the reference value, to which the determined levels of the biomarker are compared, may be a previous value obtained for a specific subject. This kind of reference value may be used if the method is to be used to monitor progression of disease or to monitor the response of a subject to a particular treatment.

The presence, or level, of the biomarker may be used to stratify patients. This stratification may be used to decide the appropriate treatment.

The method of the invention may also be used to monitor progression of an inflammatory disease, such as rheumatoid arthritis, and/or to monitor the efficacy of treatments administered to a subject. This may be achieved by analysing samples taken from a subject at various time points following initial diagnosis and monitoring the changes in the levels of the biomarker, and comparing these levels to normal and/or reference values. In this case reference levels may include the initial levels of the biomarker in the subject; or the levels of the biomarker in the subject when they were last tested, or both.

The method of the invention may also be used to determine the appropriate treatment for a subject. The method may be used to offer personalised medicine solutions. In one embodiment, the presence of the biomarker may be sufficient to result in a diagnosis of an inflammatory disorder such as rheumatoid arthritis, and may be used to indicate what the most appropriate therapy is.

Advantageously, the invention herein demonstrates that anti-cTNC5 can discriminate amongst patients with early synovial inflammation those who will go on to develop rheumatoid arthritis, and those whose disease will resolve or those who will develop a disease that is not rheumatoid arthritis.

Therefore, the presence of the biomarker of the invention in a subject/patient may indicate that they are likely to develop RA. The subject may have early synovial inflammation. The determination of the inflammatory disorder status in a subject may comprise the determination that a patient has, or is likely to develop, rheumatoid arthritis. The absence of the biomarker of the invention in a subject with early synovial inflammation may indicate that the inflammation will resolve (i.e. it is less likely that RA will occur) or that the person will not go on to develop RA. Additionally or alternatively, the method of the invention may also be used to monitor the likely efficacy of treatments to be administered to a subject for RA or another disease.

According to another aspect of the invention, there is provided a method of selecting a patient for treatment for RA, the method comprising determining the inflammatory disorder status of a subject afflicted with early synovitis according to the invention herein, wherein the presence of the biomarker of the invention indicates that the patient may be selected for treatment and/or monitored for development of RA; and optionally wherein in the absence of the biomarker of the invention the subject is not selected for further treatment and/or monitoring.

It may be appropriate if a subject has the biomarker in a sample obtained therefrom to use a therapeutically effective amount of one or more of DMARDs including methotrexate, an anti-TNF drug; an anti-IL17 therapy; a T-cell co-stimulation modulator (such as Orencia™—abatacept): an interleukin-6 (IL-6) inhibitor (such as Actemra™—tocilizumab); an anti-CD20 antibody (such as Rituxan™—rituxumab); or a B cell activating factor (such as anti-BAFF). Other alternative therapies include inhibitors of janus kinase (JAK) (such as Tofacitinib™) I, inhibitors of spleen tyrosine kinase (Syk) (such as Fostamatinib™), antiTNC antibodies or antibodies to citrullinated tenascin-C domains.

Alternatively, or additionally, the therapy may be the administration of a therapeutically effective amount of an agent that modulates the biological activity of citrullinated tenascin-C or one or more fragments of citrullinated tenascin-C, such as the biomarker. The agent may modulate the biological activity of citrullinated tenascin-C or one or more fragments thereof, such as the biomarker, in one or more of the following ways:
by altering one or more physical properties;
by altering the binding properties;
by altering the antigenicity;
by altering the level of citrullination;
by altering the ratio of citrullinated tenascin-C to non-citrullinated tenascin-C, or one or more fragments thereof, for example by altering the citrullination at one or more specific domains (e.g. the FBG domain);
by altering one or more specific citrullinated residue(s) of tenascin-C, or fragment thereof, to a non-citrullinated form of the residue(s), wherein the specific citrullinated residue(s) may be selected from any of the group comprising residues 2187, 2192, 2197 and 2198; or combinations thereof; or wherein the specific citrullinated residue(s) may be selected from any of the group comprising residues 2187, 2192, 2197, 2198 and 2200; or combinations thereof.

The agent that modulates the biological activity of citrullinated tenascin-C or one or more fragments thereof may down-regulate or up-regulate the biological activity of citrullinated tenascin-C or one or more fragments thereof.

The agent that modulates the biological activity of citrullinated tenascin-C or one or more fragments thereof may be an inhibitor of citrullination of tenascin-C; or an inhibitor of the binding properties of citrullinated tenascin-C; or a competitive binding inhibitor of citrullinated tenascin-C.

The agent that modulates the biological activity of citrullinated tenascin-C or one or more fragments thereof may be an antagonist of the TLR-4 receptor and/or the Fcγ receptor.

The agent that modulates the biological activity of citrullinated tenascin-C or one or more fragments thereof may be selected from the group consisting of short interfering RNA (SiRNA) molecules, short hairpin RNA molecules (shRNA), antisense oligonucleotides, compounds with binding affinity for citrullinated tenascin-C, antibodies (polyclonal or monoclonal) and antigen-binding fragments thereof, small inhibitor compounds, a domain of citrullinated tenascin-C or variant thereof, polypeptides and proteins. Where the agent is an antibody or antigen-binding fragment it may have specificity for Toll Like Receptor 4 (TLR4), citrullinated tenascin-C or a fragment/domain thereof; or a binding affinity for the FBG domain of citrullinated tenascin-C.

The agent that modulates the biological activity of citrullinated tenascin-C or one or more fragments thereof may modulate the biological activity of the FBG domain of citrullinated tenascin-C.

The agent that modulates the biological activity of citrullinated tenascin-C or one or more fragments thereof may modulate the activity of citrullinated tenascin-C which is citrullinated at least at the FBG domain; only at the FBG domain; or at one or more domains other than the FBG domain (e.g. the fibronectin type III like repeats). The citrullinated tenascin-C or one or more fragments of citrullinated tenascin-C may be citrullinated at one or more specific residue(s) wherein the specific residue(s) may be selected from residues 2187, 2192, 2197 and 2198. The citrullinated tenascin-C or one or more fragments of citrullinated tenascin-C may be citrullinated at two or more specific residue(s) wherein the specific residue(s) may be selected from residues 2187, 2192, 2197 and 2198. The citrullinated tenascin-C or one or more fragments of citrullinated tenascin-C may be citrullinated at three or more specific residue(s) wherein the specific residue(s) may be selected from residues 2187, 2192, 2197 and 2198. The citrullinated tenascin-C or one or more fragments of citrullinated tenascin-C may be citrullinated at one or more specific residue(s) wherein the specific residue(s) may be selected from residues 2187, 2192, 2197, 2198 and 2200. The citrullinated tenascin-C or one or more fragments of citrullinated tenascin-C may be citrullinated at two or more specific residue(s) wherein the specific residue(s) may be selected from residues 2187, 2192, 2197, 2198 and 2200. The citrullinated tenascin-C or one or more fragments of citrullinated tenascin-C may be citrullinated at three or more specific residue(s) wherein the specific residue(s) may be selected from residues 2187, 2192, 2197, 2198 and 2200. The citrullinated tenascin-C or one or more fragments of citrullinated tenascin-C may be citrullinated at four or more specific residue(s) wherein the specific residue(s) may be selected from residues 2187, 2192, 2197, 2198 and 2200.

The sample may be a sample of blood, serum, plasma, synovial fluid and/or joint tissue derived from the subject. The sample may be pre-RA sera from the subject.

Some or all of the steps of the method of the invention may be carried out in vitro.

The subject may be mammal. The mammal may be a human, but may alternatively be a monkey, ape, cat, dog, cow, horse, rabbit or rodent.

Information regarding the inflammatory disorder status of a subject may be relayed to a third party, such as a doctor, other medical professional, pharmacist or other interested party. This information may be relayed digitally, for example via email, SMS or other digital means.

According to another aspect of the invention there is provided a kit for use in determining the inflammatory disorder status of a subject comprising at least one agent for detecting the presence, or the level, of the biomarker in a sample provided by the subject.

In one embodiment the kit is for use in determining the rheumatoid arthritis status of a subject.

The kit may comprise instructions for suitable operational parameters in the form of a label or separate insert. The instructions may inform a user about how to collect the sample.

The kit may comprise the peptide of the invention and/or one or more fragments of citrullinated tenascin-C, samples to be used as standard(s) for calibration and comparison.

The kit may also comprise instructions to compare the level of the biomarker detected in a sample with a calibration sample or chart. The kit may also include instructions indicating what level of the biomarker is diagnostic of an inflammatory disorder. The instructions may indicate that the presence of any biomarker is diagnostic of an inflammatory disorder.

The biomarker of the invention may be detected together with other known biomarkers for inflammatory disorders. The peptide of the invention may be used together with other known peptides for known biomarkers for the inflammatory condition. Therefore, the kit may further comprise a panel of peptides and/or antibodies for detecting a panel of biomarkers for the inflammatory condition. The methods or uses herein described according to the invention may further comprise the use of a panel of peptides and/or antibodies for detecting a panel of biomarkers for the inflammatory condition.

Such known biomarkers may be other known peptides or auto-antibodies, such as ACPAs. For example a panel of biomarkers may be used as described in Hansson et al. (Arthritis Research & Therapy 2012, 14:R201, incorporated herein by reference), also described in table 7 herein. An example of a known biomarker is citrullinated alpha-enolase as an auto-antigen in rheumatoid arthritis. Any one or more of the peptides of table 7 herein may be selected for use with the peptide or biomarker of the invention.

According to a yet further aspect, the invention provides the use of the determination of the presence, or the level, of the biomarker in a sample obtained from a subject as a means of assessing the inflammatory disorder status in the subject. The sample may be blood, serum, plasma, synovial fluid and/or joint tissue

In a preferred embodiment the invention provides the use of the determination of the presence, or the level, of the biomarker in a blood or serum sample as a means of assessing the rheumatoid arthritis status in an individual. The sample may comprise pre-RA sera.

According to another aspect the invention provides the use of the biomarker described herein, as a biomarker for an inflammatory disorder.

According to a further aspect the invention provides the peptide or biomarker of the invention for use in a method of determining the appropriate treatment for a subject having an inflammatory disorder.

According to a further aspect the invention provides an assay comprising:
i) measuring the presence of the biomarker, in a sample from a patient who presents no symptoms or symptoms of undifferentiated synovitis for determining the likelihood of rheumatoid arthritis in the patient; and
ii) concluding if the biomarker is present in the sample, this indicates the likelihood of rheumatoid arthritis in the patient.

The assay of this aspect of the invention may include the step of measuring the level of the biomarker in a sample from a patient, and comparing the measured or quantified amount of the biomarker with a reference value, and if the amount of the biomarker is increased relative to the reference value, identifying the subject as having an increased probability of having rheumatoid arthritis. The reference value may be from a control subject who does not have rheumatoid arthritis.

The invention may provide a method of treating an inflammatory disorder in a subject comprising detecting the presence, or the level, of the biomarker in a sample from the subject and administering a treatment based on the presence, or the level, of the biomarker observed.

According to a further aspect of the invention there is provided a method of determining the appropriate treatment for a subject having an inflammatory disorder.

In one embodiment the method comprises the steps of:
(i) providing a sample derived from the subject; and
(ii) testing the sample for the presence of the biomarker wherein the presence or absence of the biomarker indicates the appropriate treatment.

In a preferred embodiment the presence of the biomarker determines that the subject should be administered an effective amount of an agent or composition, the agent or composition may be one or more of DMARDs including methotrexate, anti-TNF drug; an anti-IL17 therapy; a T-cell co-stimulation modulator (such as Orencia™—abatacept): an interleukin-6 (IL-6) inhibitor (such as Actemra™—tocilizumab); an anti-CD20 antibody (such as Rituxan™—rituxumab); a B cell activating factor (such as anti-BAFF); an inhibitor of janus kinase (JAK) (such as Tofacitinib™); an inhibitor of spleen tyrosine kinase (Syk) (such as Fostamatinib™); antiTNC antibodies or antibodies to citrullinated tenascin-C domains, and an agent that modulates the biological activity of citrullinated and/or non-citrullinated tenascin-C.

The invention may also provide a method for treating an inflammatory disorder in a subject comprising;
i) obtaining a sample from a subject;
ii) analysing the sample for the presence or absence of the biomarker;
iii) diagnosing the subject as having an inflammatory disorder if the biomarker is present; and
iv) administering an anti-inflammatory treatment to the diagnosed subject.

The inflammatory disorder may be as described herein and may be, for example, rheumatoid arthritis.

The anti-inflammatory treatment may be any treatment described herein with reference to any aspect or embodiment of the invention.

According to another aspect the invention provides a method of selecting a subject for treatment for an inflammatory disorder comprising:
i) obtaining a sample from a subject;
ii) analysing the sample for the presence or absence, or the level, of the biomarker;
iii) if the biomarker is present, or elevated relative to a normal control, selecting the subject for treatment for an inflammatory disorder.

According to a further aspect the invention provides a device for determining the inflammatory status of a subject, wherein the device is capable of emitting an external signal which is indicative of the inflammatory status of the subject. Preferably the device is capable of accepting a sample obtained from a subject, analysing the sample for the presence of the biomarker; and then emitting an external signal if the biomarker is detected in the sample. The external signal may be in the form of an audible noise, a visual change, a print out, a digital message to the user, an email to the user or a third party, or any other suitable signal.

According to another aspect of the invention, there is provided a binding member capable of specifically binding to a peptide according to the invention, or binding to a biomarker according to the invention.

In one embodiment, the binding member competes for binding with an autoantibody with specificity for an epitope of citrullinated tenascin-C or a fragment thereof wherein the epitope comprises at least three citrullinated arginine residues of 2187, 2192, 2197 and 2198. The binding member may compete for binding with an autoantibody with specificity for an epitope of citrullinated tenascin-C or a fragment thereof wherein the epitope comprises citrullinated arginine residues 2187, 2192, 2197 and 2198. The binding member may compete for binding with an autoantibody with specificity for an epitope of citrullinated tenascin-C or a fragment thereof wherein the epitope comprises at least three citrullinated arginine residues of 2187, 2192, 2197, 2198 and 2200. The binding member may compete for binding with an autoantibody with specificity for an epitope of citrullinated tenascin-C or a fragment thereof wherein the epitope comprises citrullinated arginine residues 2187, 2192, 2197, 2198 and 2200. Two antibodies bind to the same or overlapping epitope if each competitively inhibits (blocks) binding of the other to the antigen. That is, a 1×, 5×, 10×, 20× or 100× excess of one antibody inhibits binding of the other by at least 50% but preferably 75%, 90% or even 99% as measured in a competitive binding assay compared to a control lacking the competing antibody (see, e.g., Junghans et al., Cancer Res. 50:1495, 1990, which is incorporated herein by reference).

The binding member may comprise an antibody, antibody fragment or mimetic thereof. In one embodiment, the binding member is an antibody. The antibody or fragment thereof may be monoclonal. In another embodiment, the antibody or fragment thereof may be polyclonal. The antibody or fragment thereof may be mammalian. The antibody or fragment thereof may be human, or humanised. The antibody or fragment thereof may be non-human.

The binding member may have at least 10-fold higher affinity for binding to a peptide according to the invention relative to an equivalent non-citrullinated peptide. The binding member may have at least 10-fold higher affinity for binding to a citrullinated tenascin-C, or fragment thereof, biomarker according to the invention herein relative to an equivalent non-citrullinated tenascin-C or fragment thereof. An equivalent non-citrullinated peptide or tenascin-C may comprise or consist of the same sequence, but without the citrullinated arginines (i.e. they are non-citrullinated arginines).

In one embodiment, the binding member may have at least 50-fold higher affinity. In another embodiment, the binding member may have at least 100-fold higher affinity. In another embodiment, the binding member may have at least 200-fold higher affinity. In another embodiment, the binding member may have at least 500-fold higher affinity. In another embodiment, the binding member may have at least 1000-fold higher affinity.

Binding affinity may be measured by surface plasmon resonance Biacore X. The skilled person will understand that alternative affinity assays are also available.

The binding member may be an isolated binding member. The binding member may be recombinant.

According to another aspect of the invention, there is provided the use of the binding member according to the invention, for the detection of the peptide according to the invention, or detection of the biomarker according to the invention.

According to another aspect of the invention, there is provided a method of forming a complex between the peptide of the invention and anti-cTNC antibodies which are specific for the peptide of the invention, optionally wherein the method comprises contacting the peptide of the invention with a sample from a subject comprising anti-cTNC antibodies specific for the peptide.

The detection may be in a sample from a mammal, or in vivo in a mammal. The mammal may be human.

According to a further aspect of the invention peptide of the invention may be used as a detection reagent in the preparation of a product for detecting the biomarkers in a subject.

According to a further aspect of the invention there is provided the use of detection reagents in the preparation of products for detecting a biomarker in a subject, said method comprising:
obtaining a sample from the subject; and
detecting whether the biomarker is present in the sample by contacting the sample with the detection reagent capable of binding the biomarker, and detecting binding between the biomarker and the detection reagent,
   wherein the detection reagents are arranged to detect:
   (i) citrullinated tenascin-C or a fragment thereof which is citrullinated at at least two arginine residues of residue numbers 2187, 2192, 2197 and 2198; and/or
   (ii) autoantibodies with specificity for an epitope of citrullinated tenascin-C or a fragment thereof wherein the epitope comprises at least two citrullinated arginine residues of 2187, 2192, 2197 and 2198.

The detection reagents may comprise one or more peptides according to the invention herein. Additionally or alternatively, the detection reagents may comprise an antibody or antibody variant capable of binding the biomarker according to the invention.

According to a further aspect of the invention there is provided a method of detecting a biomarker in a subject, said method comprising:
obtaining a sample from the subject; and
detecting whether the biomarker is present in the sample by contacting the sample with an agent capable of binding the biomarker, and detecting binding between the biomarker and the agent,
   wherein the biomarker comprises:
   (i) citrullinated tenascin-C or a fragment thereof which is citrullinated at at least two arginine residues of residue numbers 2187, 2192, 2197 and 2198; and/or
   (ii) autoantibodies with specificity for an epitope of citrullinated tenascin-C or a fragment thereof wherein the epitope comprises at least two citrullinated arginine residues of 2187, 2192, 2197 and 2198.

In one embodiment the citrullinated tenascin-C or a fragment thereof is citrullinated at at least three arginine residues of residue numbers 2187, 2192, 2197 and 2198; and/or the epitope for the autoantibodies comprises at least three citrullinated arginine residues of 2187, 2192, 2197 and 2198. In another embodiment the citrullinated tenascin-C or a fragment thereof is citrullinated at four arginine residues of residue numbers 2187, 2192, 2197 and 2198; and/or the epitope for the autoantibodies comprises four citrullinated arginine residues of 2187, 2192, 2197 and 2198.

In an embodiment wherein the biomarker is citrullinated tenascin-C or a fragment thereof, the agent capable of binding to the biomarker may be an antibody. The antibody may be a monoclonal antibody. The antibody may be a non-human antibody, such as a non-human monoclonal antibody. The antibody may be human or humanized. The antibody may be chimeric.

In an embodiment wherein the biomarker is an autoantibody, the agent capable of binding the biomarker may comprise a peptide or a protein. The peptide or protein may be recognisable by the autoantibody. The peptide may be any one of the peptides described herein according to the invention.

The skilled man will appreciate that preferred features of any one embodiment and/or aspect of the invention may be applied to all other embodiments and/or aspects of the invention.

### Definitions

By “inflammation” we include the meaning of local accumulation of fluid, plasma proteins, and cells, such as white blood cells and local tissue resident cells, that is initiated by tissue injury, infection or a local immune response.

By “acute inflammation” we include the meaning of the initial stages (initiation) of inflammation and the short-term transient inflammatory response immediately after injury, infection or local immune response. Typically, acute inflammation is rapidly resolved, lasting from a matter of minutes to no longer that a few days.

By “chronic inflammation” we include the meaning of persistent and/or non-resolved inflammation. It is often associated with inappropriate destruction of healthy tissue. This may be progressive and last over a period of weeks or longer. Chronic inflammation is typically associated with persistent infection or disease including, but not limited to, autoimmune conditions.

By “chronic joint inflammation” we include the meaning of persistent inflammation that is progressive and unremitting over a period of weeks to months, resulting in distortion of the affected joint and radiographic evidence of cartilage and bone destruction as observed in human disease (Kelly, Harris, Ruddy and Sledge, Textbook of Rheumatology 4th Edition).

In experimental murine models, chronic joint inflammation is characterised by inflammation that does not subside and causes inappropriate tissue destruction, even over a relatively short period of time. This is characterized (and can be identified) histologically by the prolonged presence of inflammatory cells in the synovium and joint space, chondrocyte death, and cartilage and bone erosion.

By an “agent” we include all chemical entities, for example oligonucleotides, polynucleotide, polypeptides, peptidomimetics and small compounds.

By “citrullinated” we mean the conversion of one or more arginine amino acids in a protein into the amino acid citrulline.

By “a fragment of citrullinated tenascin-C” or “one or more fragments of citrullinated tenascin-C” we mean a citrullinated peptide or domain derived from citrullinated tenascin-C. The fragment of citrullinated tenascin-C may be a citrullinated FBG domain, a citrullinated TA domain, a citrullinated EGF-L domain, a citrullinated TNIII domain or any other sequence from within citrullinated tenascin-C. Preferably the fragment of citrullinated tenascin-C is antigenic. Preferably the fragment of citrullinated tenascin-C is biologically active.

By “fragment” we mean at least 10 nucleotides, for example at least 15, 16, 17, 18, 19, 20, 21, 22, 23, 24 or 25 nucleotides.

By “variant” we mean that the nucleotide sequence shares at least 90% sequence identity with the full length sequence of interest, for example at least 50%, 55%, 60%, 65%, 70%, 75%, 80%, 85%, 90%, 95%, 96%, 97%, 98% or 99% sequence identity.

The percent sequence identity between two polynucleotides may be determined using suitable computer programs, for example the GAP program of the University of Wisconsin Genetic Computing Group and it will be appreciated that percent identity is calculated in relation to polynucleotides whose sequences have been aligned optimally.

The alignment may alternatively be carried out using the Clustal W program (as described in Thompson et al., 1994, Nuc. Acid Res. 22:4673-4680). The parameters used may be as follows:

Fast pairwise alignment parameters: K-tuple(word) size; 1, window size; 5, gap penalty; 3, number of top diagonals; 5. Scoring method: x percent.

Multiple alignment parameters: gap open penalty; 10, gap extension penalty; 0.05.

Scoring matrix: BLOSUM.

Alternatively, the BESTFIT program may be used to determine local sequence alignments.

By “antibody” we include substantially intact antibody molecules, as well as chimeric antibodies, human antibodies, humanised antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same. In particular, the term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, humanised antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g., murine, antibody and the constant region of a human antibody. Methods for making humanised antibodies are described in, for example, U.S. Pat. No. 5,225,539.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment [25] which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers (PCT/US92/09965) and; (ix) “diabodies”, multivalent or multispecific fragments constructed by gene fusion (WO94/13804).

Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associated with each other to form an antigen binding site: antigen binding sites are formed by the association of the first domain of one polypeptide within the multimer with the second domain of another polypeptide within the multimer (WO94/13804).

By “autoantibody” we mean any antibody manufactured by a subject's immune system that is directed against one or more of the subject's own proteins.

An “autoantigen” is an endogenous antigen that stimulates the production of autoantibodies.

By “antigen-binding fragment” we mean a functional fragment of an antibody that is capable of binding to citrullinated tenascin-C.

“Specific binding” or “specifically binding” is generally used to refer to the situation in which the binding member will not show any significant binding to molecules other than its specific binding partner(s)/epitope, and, e.g., has less than about 30%, preferably 20%, 10%, or 1% cross reactivity with any other molecule.

The term “subject” means all animals including humans. Examples of subjects include humans, cows, dogs, cats, goats, sheep, and pigs. The term “patient” means a subject having a disorder in need of treatment.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

Advantageously, the invention herein demonstrates that anti-cTNC5 can discriminate amongst patients with early synovial inflammation those who will go on to develop RA, and those whose disease will resolve or who will develop disease that is not RA.

Therefore, the presence of cTNC5 or anti-cTNC5 in a subject/patient may indicate that they are likely to develop RA. The subject may have early synovial inflammation. The determination of the inflammatory disorder status in a subject may comprise the determination that a patient is likely to develop reactive arthritis. The absence of cTNC5 or anti-cTNC5 in a subject with early synovial inflammation may indicate that the inflammation will resolve (i.e. it is less likely that RA will occur).

Examples embodying an aspect of the invention will now be described with reference to the following figures:

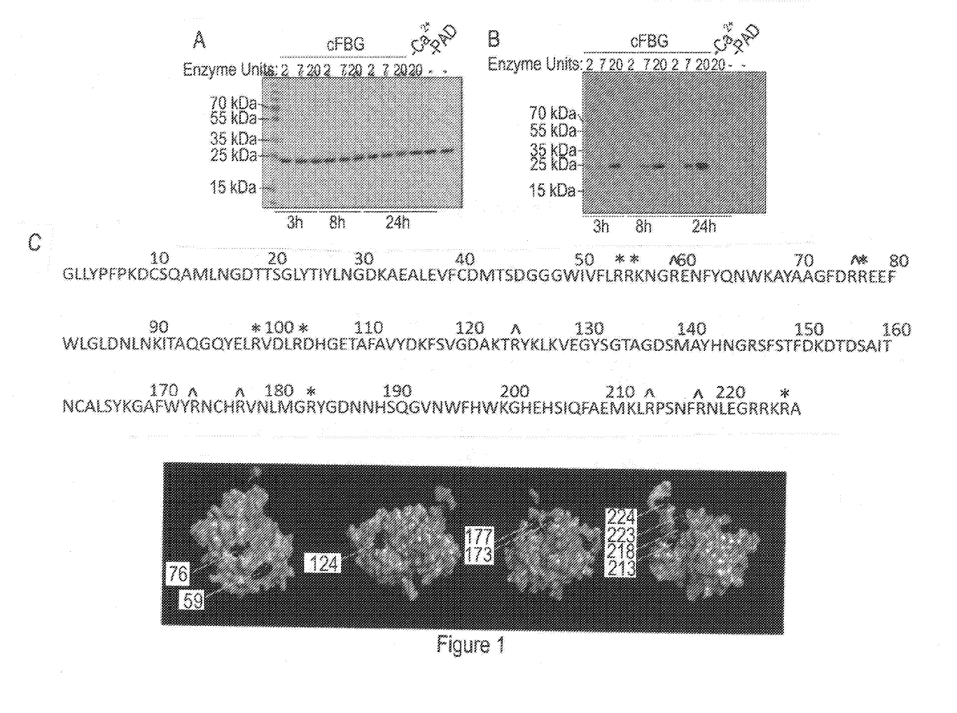

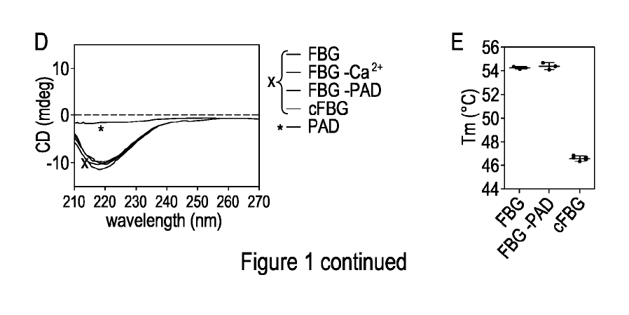

FIG. 1: Citrullination of FBG by rPAD2.

Coomassie stained SDS gels (A) and immunoblot probed with an AMC antibody (B) of FBG citrullinated by rPAD2. FBG incubated in buffer with rPAD2 but without Calcium (—Ca²⁺) or without enzyme (-PAD) served as negative controls. Untreated native FBG was loaded in the last well. (C) Arginine residues citrullinated by rPAD2, hPAD2 and hPAD4 were determined by LC-MS/MS. Arginine residues that were modified to citrulline are highlighted in blue (marked̂), all non-citrullinated arginines are shown in green (marked *). (D) The super secondary structure of native and citrullinated FBG (3 hours, 20 U rPAD2/mg FBG) was analysed by circular dichroism. (E) The melting temperature of native and citrullinated FBG (3 hours, 20 U rPAD2/mg FBG) was analysed by differential scanning fluorimetry. Results are shown as mean+/−SD from 3 independent experiments.

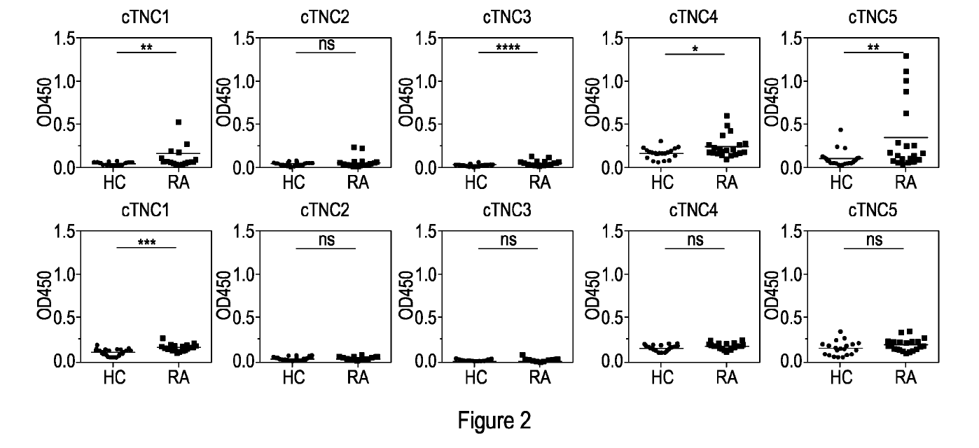

FIG. 2: Identifying the citrullinated antibody epitope.

IgG response to citrullinated FBG peptides (cTNC) and arginine containing control peptides (rTNC) in patients with rheumatoid arthritis (RA; n=20) and healthy controls (HC, n=20). Mann-Whitney U test was used to calculate p values for differences between groups (ns=no significant difference, *=p<0.05 and **=p<0.01, ***=p<0.001, ****=p<0.001).

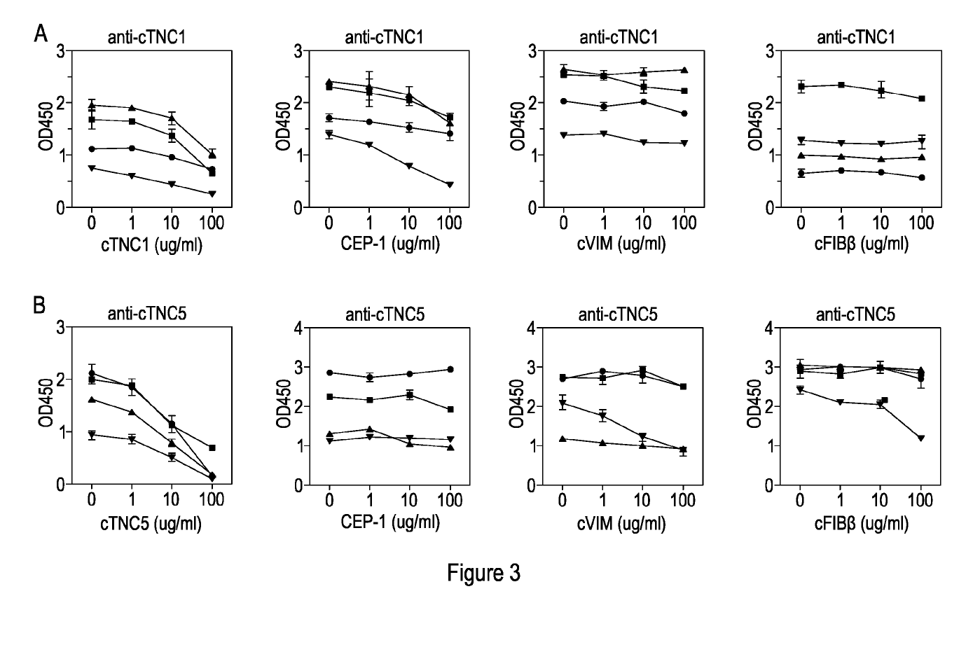

FIG. 3. Anti-cTNC antibody cross-reactivity with CEP-1, cVIM and cFIBβ.

Sera, double-reactive with peptides cTNC1 or cTNC5 and CEP1, cVIM or cFIBβ3 respectively, were pre-incubated with increasing concentrations of the indicated peptides, and the IgG response to cTNC1 (A) and cTNC5 (B) was measured.

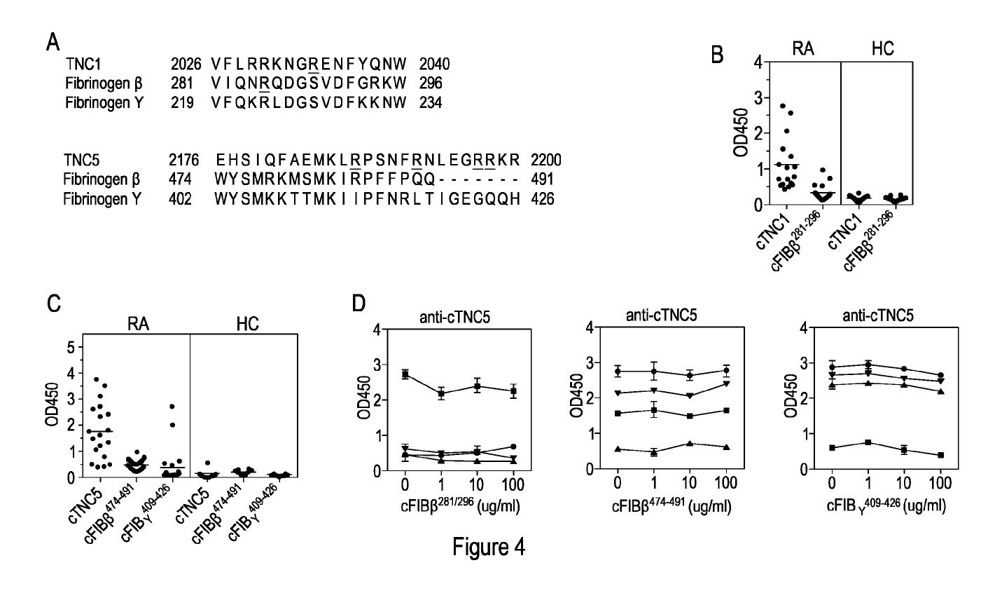

FIG. 4: Anti-cTNC antibody cross-reactivity with homologous fibrinogen peptides.

(A) Multiple Sequence Alignment (Clustal Omega) of Tenascin-C, fibrinogen β chain and fibrinogen γ chain. Arginines found citrullinated in vitro are highlighted in red, citrullinated arginines described as ACPA epitopes are underlined. (B and C) IgG response to cTNC1, cTNC5 and homologous citrullinated fibrinogen peptides was measured in sera positive for cTNC1 (B) or cTNC5 (C). (D) Sera, double-reactive to peptides cTNC1 or cTNC5 and the homologous fibrinogen peptides were pre-incubated with increasing concentrations of the indicated peptides, and IgG response to cTNC1 and cTNC5 was measured.

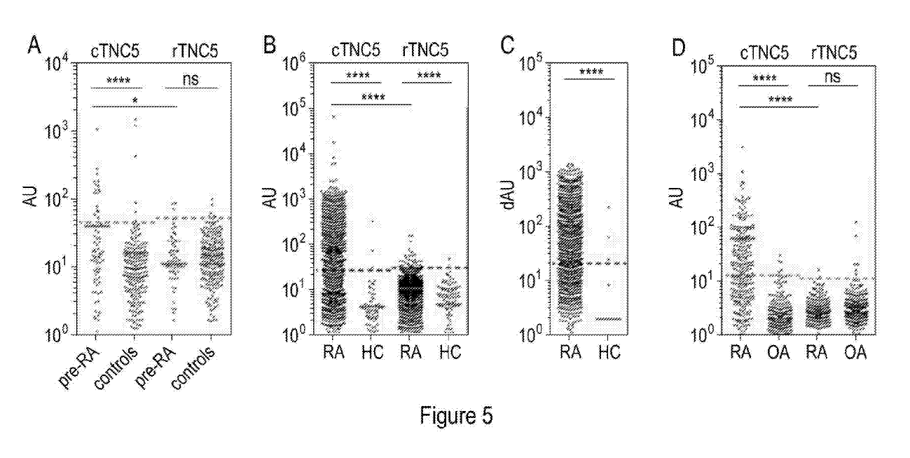

FIG. 5: Anti-cTNC5 antibody response in RA and pre-RA sera.

IgG response to cTNC5 and rTNC5 in serum samples from (A) 101 pre-RA individuals (pre-RA) and 326 controls, and (B) from 1985 RA patients (RA) and 150 healthy controls (HC) from the EIRA cohort. (C) IgG response to cTNC5 in the EIRA cohort in dAU when controlled for binding to arginine control peptide. dAU was calculated as described in Material and Methods. (D) IgG response to cTNC5 and rTNC5 in 287 serum samples from RA patients (RA) and 330 serum samples from osteoarthritis patients (OA) from an U.S. cohort. The red line indicates the mean. Blue dotted lines indicate cut-off for positivity. Mann-Whitney U test was used to calculate p values for differences between groups (n.s.=no significant difference, ****=p<0.001).

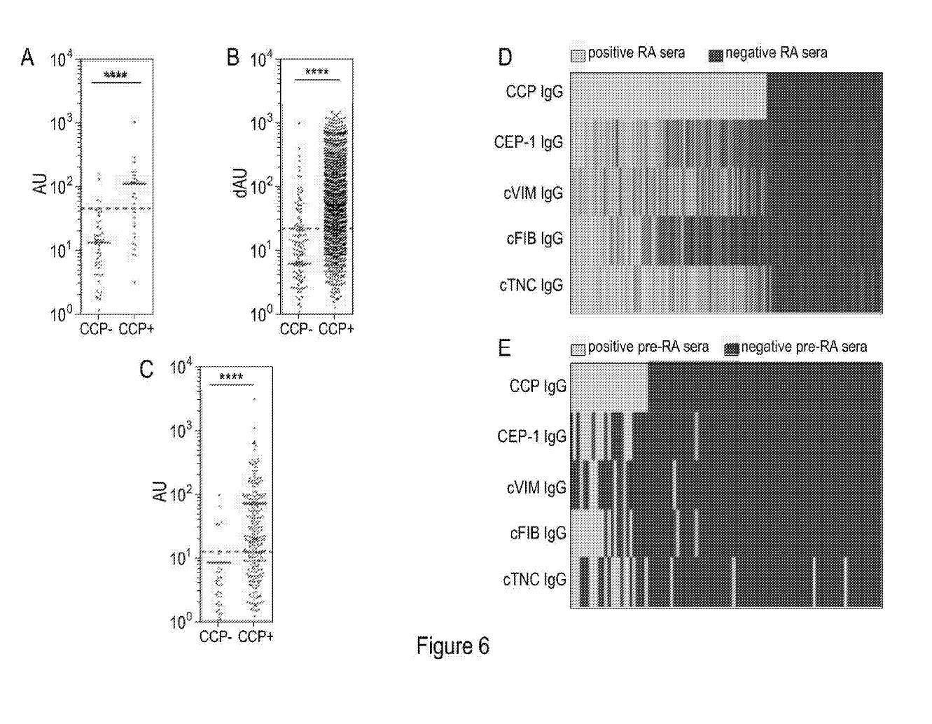

FIG. 6: Anti-cTNC antibody levels correlate with CCP2 levels.

IgG response to cTNC5 in (A) CCP2 positive (CCP2+, n=1255) and CCP2 negative (CCP2−, n=730) sera of RA patients of the EIRA cohort, (B) CCP2 positive (n=240) and CCP2 negative (n=47) sera of RA patients of the U.S. cohort and (C) in CCP2 positive (n=27) and CCP2 negative (n=74) sera of pre-RA individuals. (D and E) Heat maps showing the presence (blue) or absence (red) of an IgG antibody response to ACPA epitopes in RA sera from the EIRA cohort (D) or pre-RA sera (E).

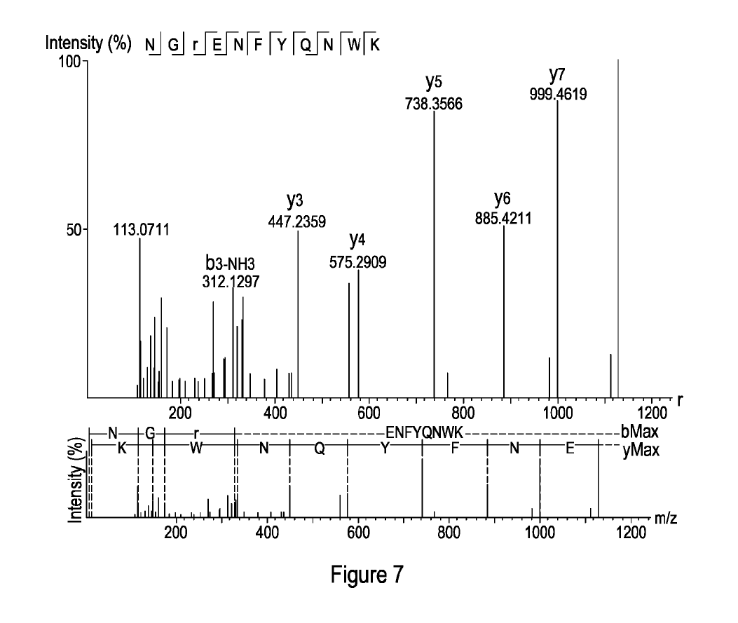

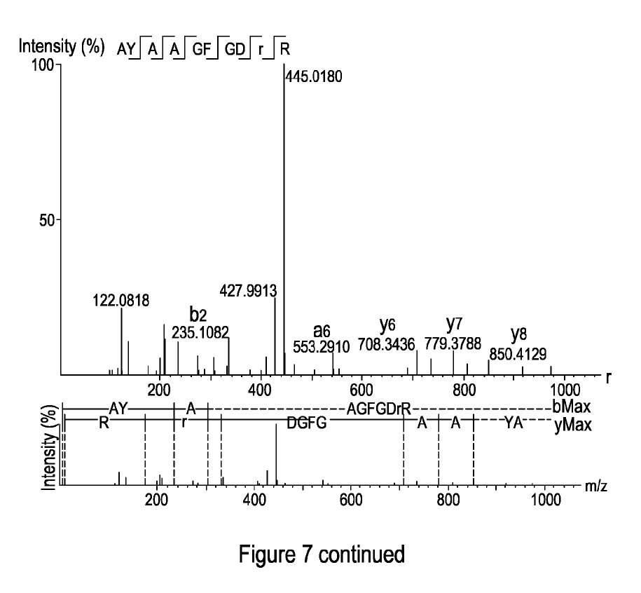

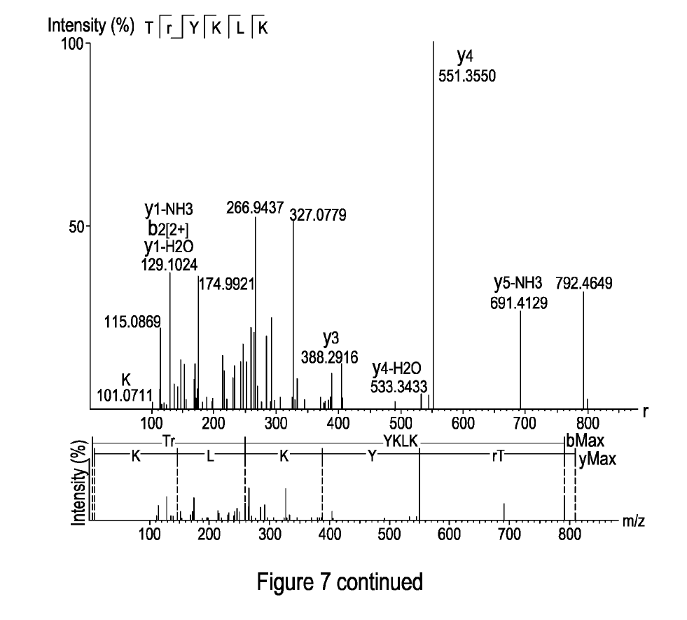

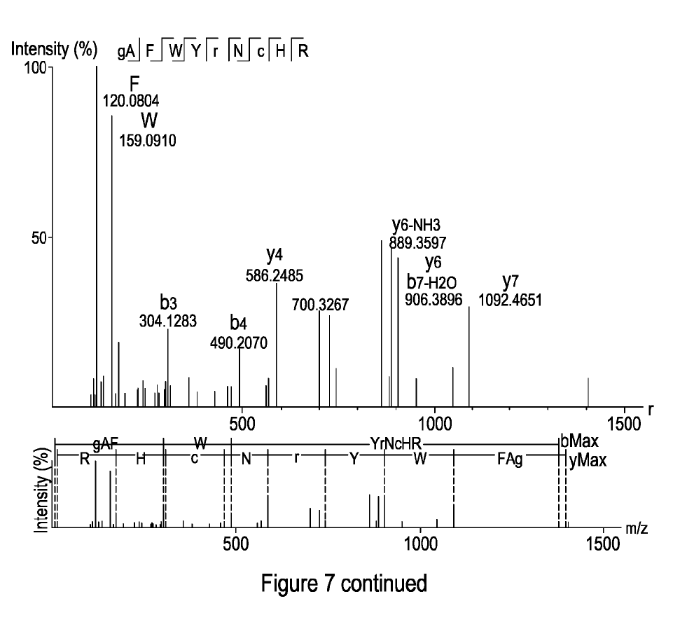

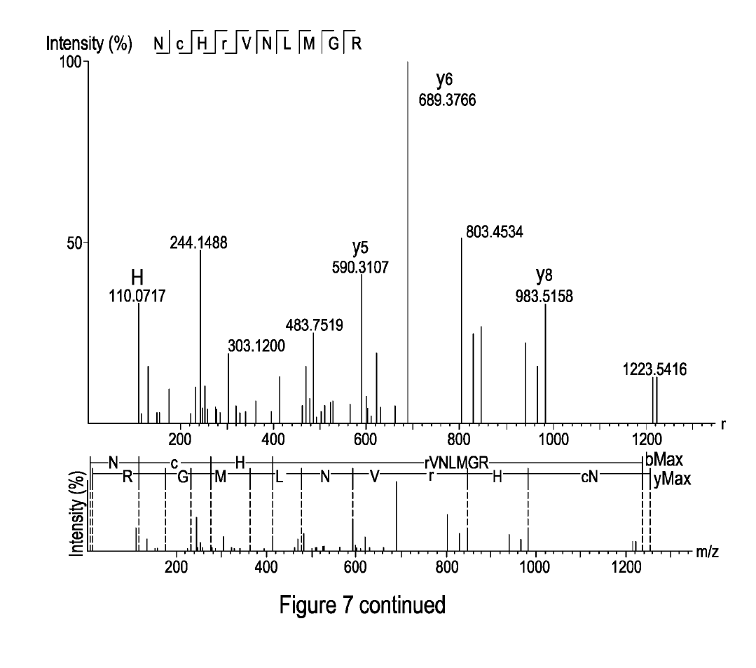

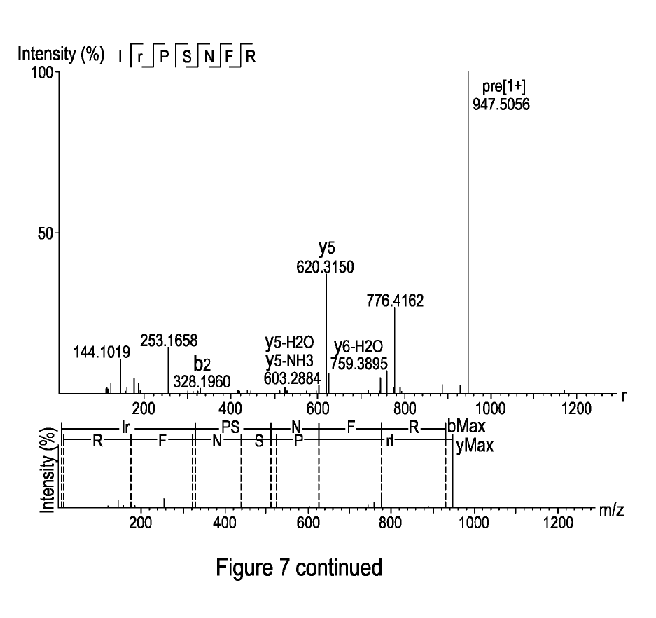

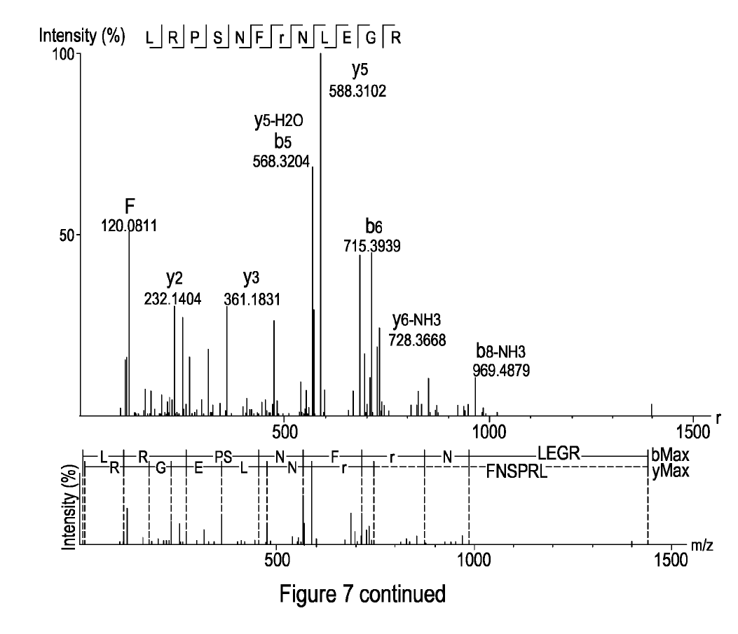

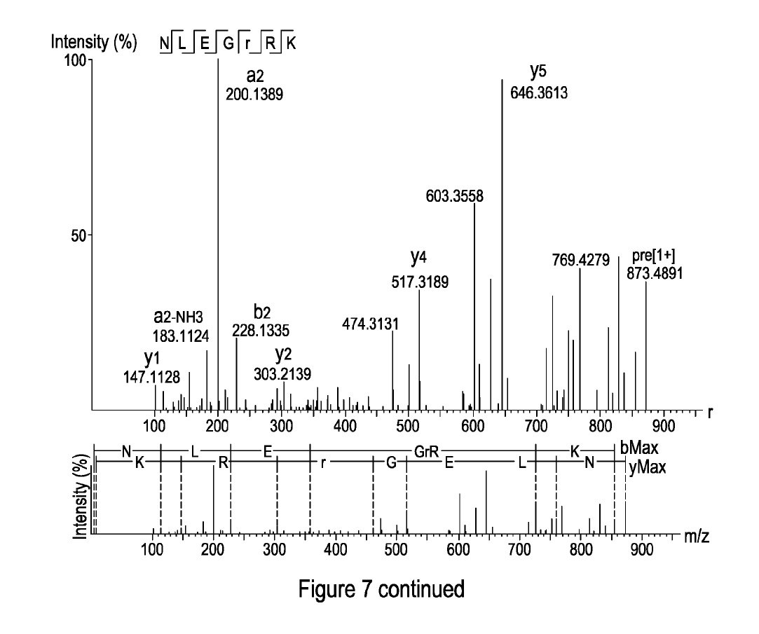

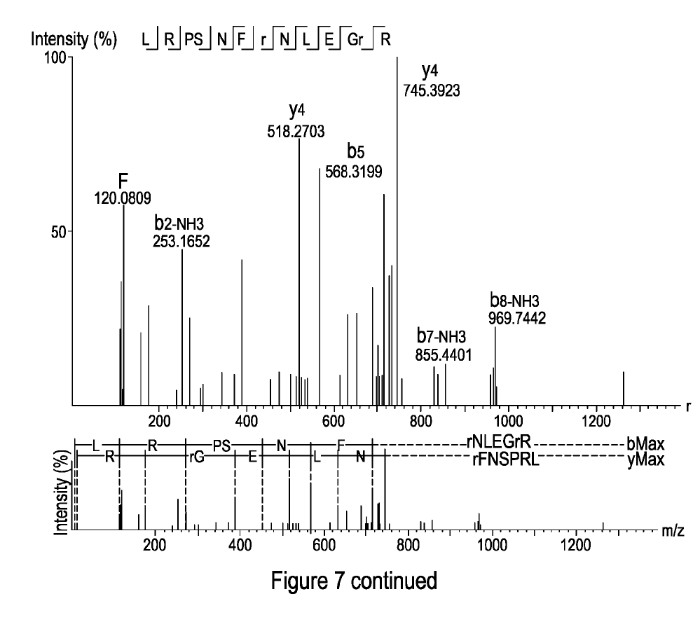

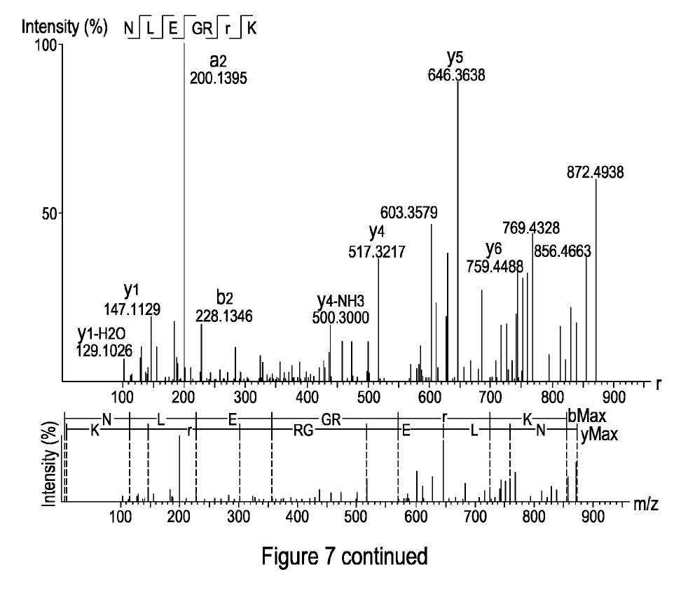

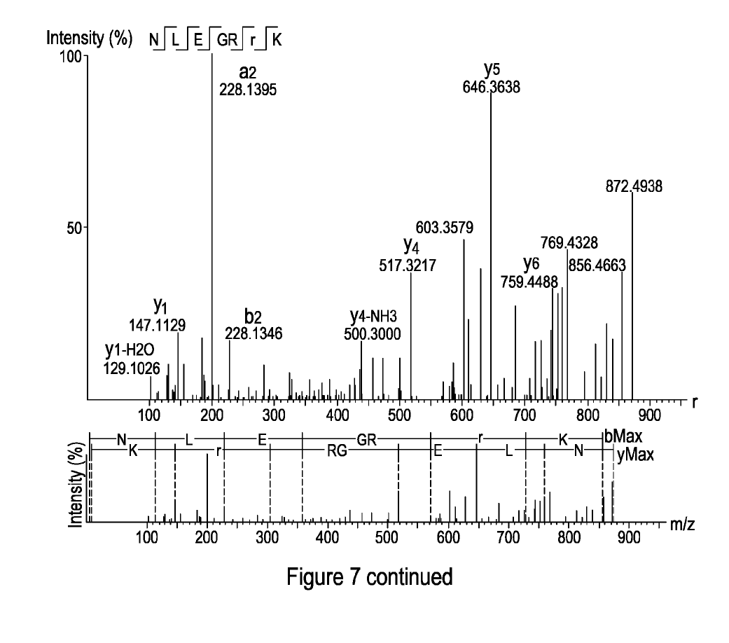

FIG. 7: Mass spectrometry analysis of citrullinated peptides derived from the FGB domain of Tenascin C (accession number P24821). The MS/MS spectra of tryptic peptides 2031-2041, 2042-2051, 2097-2012, 2143-2151, 2148-2157, 2186-2192, 2186-2197, 2193-2199 and 2186-2198 are shown, where r refers to the citrullinated position, indicating that R2033, R2050, R2098, R2147, R2151, R2187, R2192, R2197 and R2198. The matched fragment ions of the y-type (red) and b-type (blue) are shown. The MS/MS spectrum in the bottom panel indicates matches of predominant fragment ions to the peptide sequence.

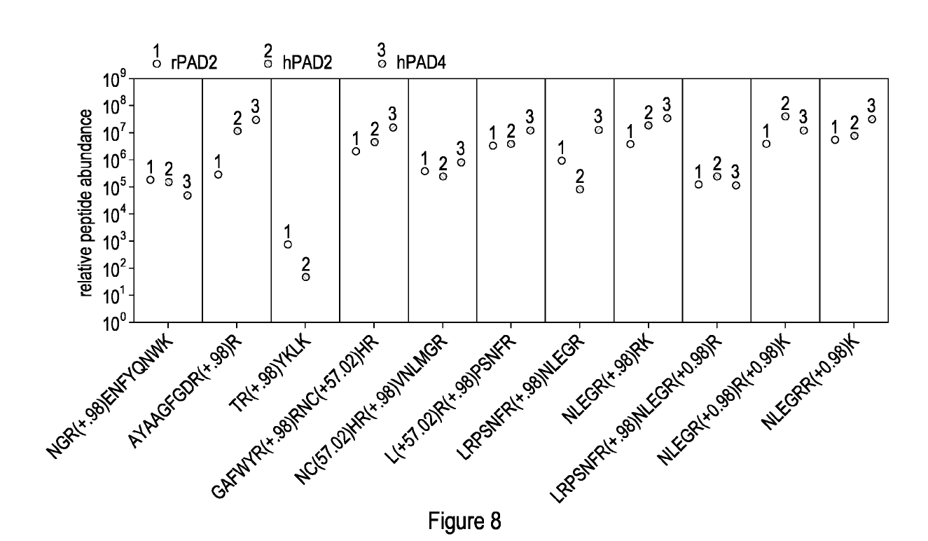

FIG. 8: Relative quantitation of citrullinated peptide occurrence in FBG samples citrullinated with 2 Units of rPAD2, hPAD2 and hPAD4 for 24 hours. (No peptide TR(+0.98)YKLK was detected in the sample citrullinated with hPAD4, however this peptide was detected when citrullinated with 20 U of enzyme, data not shown).

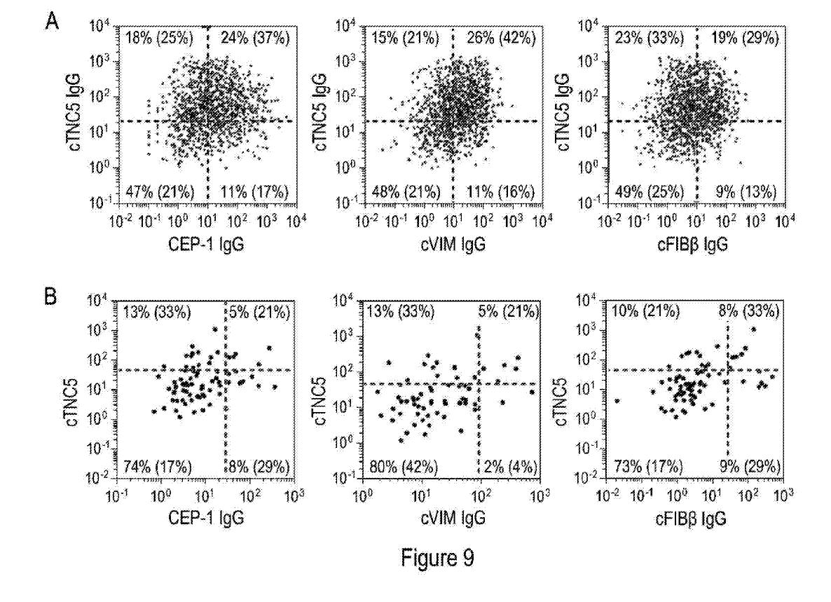

FIG. 9: Correlation of anti-cTNC5 IgG levels with IgG levels of other ACPA in RA (A) and pre-RA (B) sera. Numbers in corners are percentage of positivity; numbers in parenthesis are percentage of positivity within the CCP2-positive group. The dotted lines indicate cut-off for positivity.

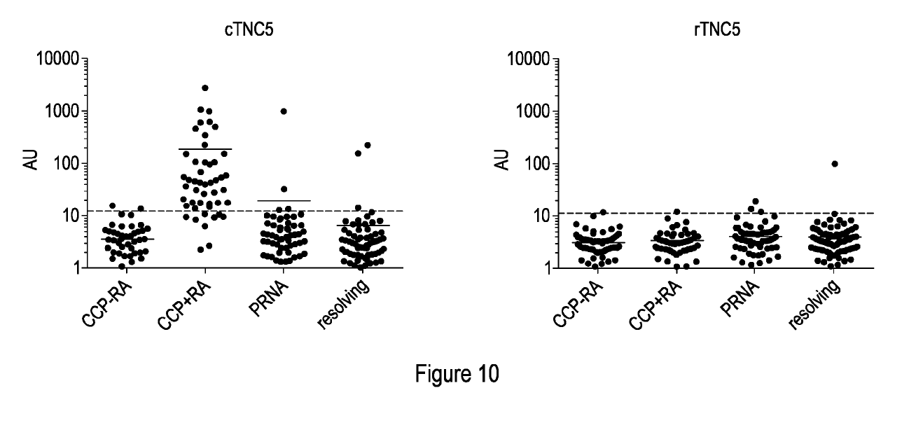

FIG. 10: Anti-cTNC5 antibody response in sera from patients with early synovitis. IgG response to cTNC5 and rTNC5 in serum samples from people with CCP negative RA (CCP− RA)(n=53), CCP positive RA (CCP+ RA)(n=48), persistent non-RA arthritis (PNRA)(n=66) and resolving RA (resolving)(n=96). The solid line indicates the mean, and the black dotted lines indicate the cut-off for positivity. AU, arbitrary units; cTNC, citrullinated tenascin-C; rTNC, non-citrullinated tenascin-C.

### IDENTIFICATION OF AN IMMUNODOMINANT PEPTIDE FROM CITRULLINATED TENASCIN-C AS A MAJOR TARGET FOR AUTOANTIBODIES IN RHEUMATOID ARTHRITIS

It was investigated whether citrullinated tenascin-C(cTNC), an extracellular matrix protein expressed at high levels in the joints of rheumatoid arthritis (RA) patients, is a target for the autoantibodies in RA.

Citrullinated sites were mapped by mass spectrometry in the fibrinogen-like globe (FBG) domain of tenascin-C treated with peptidyl arginine deiminases (PAD) 2 and 4. Antibodies to cyclic peptides containing citrullinated sites were screened in sera from RA patients by ELISA. Potential cross-reactivity with well-established anti-citrullinated protein antibody (ACPA) epitopes was tested by inhibition assays. The autoantibody response to one immunodominant cTNC peptide was then analysed in 101 pre-RA sera (median 7 years before onset) and two large independent RA cohorts.

Nine arginine residues within FBG were citrullinated by PAD2 and PAD4. Two immunodominant peptides cTNC1 (VFLRRKNG-cit-ENFYQNW) and cTNC5 (EHSIQFAEMKL-cit-PSNF-cit-NLEG-cit-cit-KR) were identified. Antibodies to both showed limited cross-reactivity with ACPA epitopes from a-enolase, vimentin and fibrinogen, and no reactivity with citrullinated fibrinogen peptides sharing sequence homology with FBG. cTNC5 antibodies were detected in 18% of pre-RA sera, and in 47% of 1,985 Swedish RA patients and 51% of 287 North American RA patients. The specificity was 98% compared to 160 healthy controls and 330 osteoarthritis patients.

There are multiple citrullination sites in the FBG domain of tenascin-C. Amongst these, one epitope is recognized by autoantibodies that are detected years before disease onset, and which can serve as a useful biomarker to identify ACPA-positive patients with high sensitivity and specificity in established disease.

The arginine rich domain of tenascin-C as a novel autoantigen is characterised herein by epitope mapping the FBG domain with a screening panel of RA sera, and examining the antigen specificity of two immunodominant epitopes by inhibition studies. An ELISA assay was standardised and used it to detect antibodies in pre-RA serum samples and two large independent patient cohorts with early and established RA.

All RA cases fulfilled the 1987 American College of Rheumatology (ACR) classification criteria [32]. 4 cohorts were examined, all from previously published studies with informed consent and ethical approval. (i) The screening cohort comprised 20 British RA patients and 20 healthy individuals[13]. (ii) The 101 pre-RA cases and 326 matched controls were identified in a nested case-control study in four Southern European cohorts[33]. (iii) 1,985 cases of RA and 160 controls were from the Swedish population-based case-control study EIRA (Epidemiological Investigation of RA)[34]. (iv) 287 patients with RA and 330 control patients with osteoarthritis (OA) were from the United States[35].

### Citrullination Reaction

Recombinant human FBG[26] was incubated with rabbit skeletal muscle PAD2 (rPAD2), or recombinant human PAD2 or PAD4 (hPAD2, hPAD4), resolved on a SDS gel and stained with Coomassie-blue or Western blotted with a monoclonal human anti-modified citrulline (AMC) antibody (Modiquest Research, clone C4, 1:500).

### LC-MS/MS Analyses

Citrullinated FBG was resolved on a 12% SDS gel, Coomassie-stained protein bands were excised and in gel-digestion performed as described[36]. Peptides were analysed by LC-MS/MS.

### Peptides, ELISAs and Cross-Reactivity Assay

ELISAs were used to detect antibodies against citrullinated peptides in human sera as described[13]. Briefly, 96-well plates were coated with 10 μg/ml peptide, blocked with 2% BSA and incubated with sera diluted 1:100. Bound antibodies were detected with an HRP conjugated anti-human IgGFc monoclonal antibody (Jackson—for EIRA study, Stratech—for all other ELISAs). A standard curve of positive sera was used to calculate relative antibody titres in arbitrary units (AU) for each sample. Subtraction of the OD450 of the native peptide from the OD450 of the citrullinated peptide was used to correct reactivity and dOD450 values were transformed into arbitrary units using the standard curve (dAU).

### EIRA Cohort

EIRA controls were randomly selected from the population registry, to match EIRA cases on age-, gender- and residential area [34]. Smoking data was collected by questionnaire at baseline. Subjects were categorized as ever-smokers or never-smokers [34]. HLA-DRB1 subtyping and genotyping of the protein tyrosine phosphatase gene (PTPN22 rs2476601) was described before [57,58].

### Citrullination Reaction

Recombinant human FBG [26] was incubated with rabbit skeletal muscle PAD2 (rPAD2) (Sigma Aldrich), or recombinant human PAD2 or PAD4 (hPAD2, hPAD4) (Modiquest Research) in citrullination buffer (50 mM Tris pH 8, 150 mM NaCl, 10 mM CaCl₂) with 5 mM DTT for 3, 8 or 24 hours at 37° C. 1 μg of each sample was resolved on a 12% SDS gel and stained with Coomassie-blue or Western blotted with a monoclonal human anti-modified citrulline (AMC) antibody (Modiquest Research, clone C4, 1:500), following chemical modification of the membrane (0.0125% FeCl₃, 2.3 M H₂SO₄, 1.52 M H₃PO₄, 0.25 M Acetic Acid, 0.25% 2, 3-butanedione monoxime, 0.125% antipyrine).

### LC-MS/MS Analyses

Peptides were analysed on a LC-MS/MS workflow comprising a Dionex Ultimate 3000 nLC system coupled to a Q-Exactive mass spectrometer (Thermo Scientific) [59,51]. Briefly, chromatographic separation was achieved using a 50 cm nEASY spray column (Thermo Scientific, (PepMAP C18, 75 m×500 mm, 2 m particle size) and a linear acetonitrile gradient from 2-35% in 5% DMSO and 0.1% formic acid. Precursor peptides were detected with a resolution of 70000 at 200 m/z followed by the selection of up to 15 precursor ions. Raw data was imported into Progenesis QI (Waters, V4.1.4832.42146) for label-free quantitation and alignment, and peptides were identified with PEAKS (Bioinformatics Solutions, V7). The abundance of citrullinated peptides was normalised to the abundance of their non-citrullinated counterparts, which value was further normalised to the citrullinated peptide abundance detected in a non-citrullinated FBG control sample.

### Differential Scanning Fluorimetry and Circular Dichroism

Five μM FBG was mixed with 5×SYPRO® orange protein gel stain (Life Technologies) in citrullination buffer and melting curves from 38° C. up to 99° C. recorded on the Applied Biosystems ViiA 7 Real-Time PCR System (excitation 550 nm, emission, 586 nm). The melting temperature (Tm) of each sample was defined as the maximum of the first derivate of the fluorescence. Circular dichroism spectra of FBG samples (200 μg/ml) were recorded using a Jasco J-815 CD Spectrometer, between 210 and 270 nm.

### Peptides

Peptides from tenascin-C(accession number P24821) and fibrinogen b (P02675) and g (P02679) chains (Table 1 and 2) were synthesized at a purity >90-95%, with C- and N-terminal cysteines (Pepceuticals) and solubilized in water at 10 mg/ml. CEP-1, cVIM and cFIBP peptides used for cross-reactivity assays are described elsewhere[33].

### Crossreactivity ELISA

Cross-reactivity was analysed in human sera that were reactive to both cTNC1 or cTNC5 and CEP-1, cVIM, cFIBβ or citrullinated homologous fibrinogen peptides. Sera were diluted 1:100, incubated with 1, 10 and 100 μg/ml of peptides for 2 hours, centrifuged at 10,000 g for 10 min and the supernatant added to peptide-coated plates for analysis by ELISA as described in Material and Methods.

### Statistical Analysis and Software

Mann-Whitney U tests were used to calculate p values for differences between groups (GraphPad Prism). The 98% percentile of healthy control samples was used to define the cut-off for positive antibody levels. Chi-square tests were used to compare frequency distributions of categorical variables while t-tests and Wilcoxon rank-sum tests were used to compare all continuous variables. To determine the association of smoking, HLA-DRB1 SE (subtypes DRB1*01 (except DRB1*0103), DRB1*04 and DRB1*10), HLA-DRB1*13 and PTPN22 with different RA subsets, odds ratios (OR) with 95% Confidence Intervals (95% CI) were calculated using unconditional logistic regression models, with unexposed cases and controls as reference group. Analyses were adjusted for age, gender and residential area. All analyses were performed using SAS version 9.3. Heatmaps were generated using G-ProX and three-dimensional models were created using Pymol (Schrodinger, LLC).

### FBG is Citrullinated In Vitro by PAD2 and PAD4

FBG was citrullinated by rPAD2, demonstrated by a small increase in the molecular weight on Coomassie-stained SDS-PAGE and Western Blotting with an AMC antibody (FIG. 1A, B). Mass spectrometry analysis of citrullinated FBG covered 14 of the 17 arginines present in this domain of tenascin-C, of which 9 were citrullinated (FIG. 1C, FIG. 7). rPAD2, hPAD2 and hPAD4 each citrullinated the same sites within FBG with no major difference in the degree of citrullination observed (FIG. 8). Circular dichroism showed comparable spectra between native FBG and citrullinated FBG (FIG. 1D) indicating that citrullination of FBG does not impact the secondary structure of the protein. Differential scanning fluorimetry however revealed a significantly lower melting temperature of citrullinated FBG (46.5+/−0.2) compared to FBG (54.3+/−0.1) (FIG. 1E), demonstrating that citrullination leads to partial protein unfolding.

cTNC1 and cTNC5 are the Primary Epitopes Recognised by ACPA in RA Patients

Five tenascin-C cyclic peptides encompassing the citrullinated residues identified by mass-spectrometry, together with their arginine-containing controls (Table 1), were used to map antibody response in a screening panel of serum samples from 20 patients with RA, and from 20 healthy subjects, by ELISA. Antibodies to citrullinated tenascin-C (cTNC) peptides cTNC1 and cTNC5 were detected in serum samples from 35% and 40% of patients respectively, but not in control sera, with no response against the arginine-containing control peptides (rTNC). There was little or no reactivity with the other three peptides tested (FIG. 2). Therefore cTNC1 and cTNC5 were selected for further study.

Anti-cTNC Antibodies Show Limited Cross-Reactivity with Other ACPA Epitopes

To examine epitope specificity and potential cross-reactivity of anti-cTNC antibodies with already identified ACPA antigens, inhibition experiments were performed with the well-defined peptides of CEP-1 (⁵KIHA-cit-EIFDS-cit-GNPTVE²¹), cVIM (⁵⁹VYAT-cit-SSAV-cit-L-cit-SSVP⁷⁴) and cFIBP (³⁶NEEGFFSA-cit-GHRPLDKK⁵²). Absorption by the homologous peptides was more efficient for cTNC5 than cTNC1. There was no cross-reactivity between anti-cTNC1 and cVIM and cFIBβ3, though there was some inhibition by the CEP-1 peptide (17 to 70% inhibition) (FIG. 3A). In contrast, there was no cross-reactivity between anti-cTNC5 and CEP-1, while these antibodies showed limited cross-reactivity with cVIM and cFIBβ3 in one serum sample (inhibition by 58% and 50%, respectively) (FIG. 3B).

Because the FBG domain of tenascin-C exhibits some sequence homology with fibrinogen we also analysed whether anti-cTNC antibodies cross-react with epitopes on citrullinated peptides containing similar sequences of fibrinogen β chain (cFibβ²⁸¹⁻²⁹⁶) and fibrinogen γ chain (cFibβ⁴⁷⁴⁻⁴⁹¹, cFibγ⁴⁰⁹⁻⁴²⁶) (FIG. 4A). From 17 sera reactive with cTNC1, 7 also reacted with cFibβ²⁸¹⁻²⁹⁶ (FIG. 4B), from 19 sera reactive with cTNC5, 14 also reacted with cFibβ⁴⁷⁴⁻⁴⁹¹, and 9 with cFibγ⁴⁰⁹⁻⁴²⁶ (FIG. 4C). To examine whether this dual positivity was due to true cross-reactivity, 4 samples that were double positive for cTNC and citrullinated fibrinogen peptide IgG were tested by inhibition experiments. No reduced reactivity to cFBG epitopes was observed when sera were pre-incubated with citrullinated fibrinogen β and γ peptides (FIG. 4D).

Anti-cTNC5 is Detected in Pre-RA Sera and with Moderate-to-High Sensitivity in Early and Established RA

In 101 pre-RA sera (median 7 years before diagnosis), 18% of pre-RA sera were positive for anti-cTNC5 antibodies (FIG. 5A) compared to 2% of 326 sera from controls. No antibodies against cTNC1 were detected. Therefore, because cTNC5 appeared to have better antigen specificity in our absorption experiments and a higher frequency of antibodies in both pre-RA and established RA, further analysis focused on cTNC5.

In the EIRA cohort, 47% of 1,985 RA sera and 2% of 160 healthy control sera were positive for antibodies to cTNC5, indicating a diagnostic sensitivity of 47% and specificity of 98%. Within the RA sera, 2.5% also bound the arginine-control peptide rTNC5 (FIG. 5B), and when controlling for binding to the arginine control peptide the sensitivity remained moderately high at 41% (FIG. 5C). This figure was higher than the frequency of citrulline-specific antibodies to CEP-1 (35%), anti-cVIM (37%) and anti-cFIBβ3 (37%), measured in the same cohort by ELISA[14].

We confirmed the moderate-to-high diagnostic sensitivity of cTNC5 (51%) in an independent US cohort of 287 sera from RA patients and 330 sera from osteoarthritis disease controls (FIG. 5D). In this cohort the binding to rTNC5 was not increased compared to OA.

Anti-cTNC5 reactivity was significantly higher in CCP2 positive patients compared to CCP2 negative patients in both RA (FIG. 6A, B) and pre-RA samples (FIG. 6C). Anti-cTNC5 antibody largely overlapped with other ACPA (FIG. 6D, E, FIG. 9) in the RA and pre-RA cohort. However in the EIRA cohort 5.4% of the serum samples exclusively reacted with the cTNC5 peptide alone. In the anti-CCP2 negative samples 4.9% reacted with cTN5.

In EIRA cTNC5 positive RA was associated with smoking (OR 1.65 vs 1.26) and HLA-DRB1 shared epitopes (OR 4.98 vs 1.68), but not with PTPN22 (OR 1.77 vs 1.44) when compared to the cTNC5 positive/CCP2 negative RA subset (Table 9). We also analysed whether cTNC5 antibodies are associated with specific HLA-DRB1 epitopes and found that cTNC5 antibodies did not associate with DRB1*10 alleles, but with HLA-DRB1*01 and DRB1*04 alleles (Table 4). Antibodies against cTNC5 negatively associated with HLA-DR13 (Table 5).

In the US cohort, cTNC5 antibody positivity was significantly associated with disease activity (DAS 28-CRP), but did not associate with other analysed clinical parameters (disease duration, swollen and tender joints, sharp score and erosion score) (Table 6).

In this study, we describe a novel citrullinated peptide from the FBG domain of tenascin-C. The citrullinated residues can be generated by either PAD2 or PAD4, yielding epitopes that are recognised by antibodies in approximately 1 of every 5 individuals with pre-clinical RA and with a moderate-to-high diagnostic sensitivity in early and established disease. Inhibition assays and analysis of antibodies to other well-characterised peptides indicate that anti-cTNC5 antibody status is independent of reactivity to other citrullinated peptides. Even though a large number of antigenic citrullinated peptides have been described as reactive with ACPA in previous reports, few have been examined with the stringent criteria used in this study. Therefore, our findings suggest that cTNC5 is a novel and independent addition to the relatively small number of citrullinated peptides which are genuinely targeted by ACPA, and which may have a role in both clinical diagnosis and investigating pathogenesis in RA.

The FBG domain of tenascin-C was citrullinated in vitro by PAD2 and PAD4. Whilst these enzymes have different substrate specificities[37], both modified the same 9 arginines in FBG to a similar degree. Lack of citrullination of 5 other arginines in FBG by any PAD reflects the specificity of this modification, likely due to hindered accessibility of these residues, or unfavourable neighbouring amino acids. Citrullinated arginines were located at 5 distinct sites within FBG, of which two, cTNC1 and cTNC5, were reactive with sera from RA patients. However, antibodies to only one, cTNC5, were detected in sera of pre-RA cases.

The different associations of cTNC1 and cTNC5 suggest that cTNC5 may be important in priming the ACPA response, whereas antibodies to cTNC1 may arise as a result of epitope spreading in more established disease. These data also reflect that the autoantibody response in RA is not citrulline-specific; instead it depends on the whole epitope around the modified residue including neighboring amino acids and the three dimensional structure[10]. It is well documented that distinct ACPA responses to different citrullinated epitopes within one protein exist, as described for example for citrullinated α-enolase[13] or fibrinogen[38]. The peptide sequence of cTNC5 is predicted to form a very distinct, exposed structure at the very C-terminus of tenascin-C, potentially rendering it more easily accessible to ACPA than cTNC1. In addition, four sites are citrullinated within TNC5, compared to only a single citrullinated site within cTNC1, which may also contribute to the higher frequency of cTNC5 ACPA observed. The frequency of anti-cTNC5 antibodies in the pre-RA cohort (18%) is comparable to antibody frequencies described for other ACPA in the same cohort, including cFIBβ (18%) and CEP-1 (15%)[33]. Analysis of a large cohort of patients with early RA, demonstrated moderate-to-high sensitivity of RA samples for cTNC5 (47%). This is the highest recorded sensitivity for any single antigenic peptide in this cohort, in this case compared to a 35%-37% sensitivity for antibodies to each of the three other antigenic peptides[14]. We also found reactivity to cTNC5 at a similar frequency (51%) in a second cohort of RA sera from U.S. patients.

ACPA generally show limited cross-reactivity[14,39]. In line with these reports, we showed that antibodies to cTNC exhibited little cross-reactivity with citrullinated enolase, vimentin and fibrinogen peptides, and are distinct from antibodies reacting with peptides from homologous regions in fibrinogen. cTNC5 ACPA positive sera were mostly found within the anti-CCP2 antibody positive RA population with cTNC5 antibody levels highest in the anti-CCP2 antibody positive subgroup, as described for other ACPA[14]. 4.9% of RA patients within the anti-CCP2 antibody negative group were also anti-cTNC5 ACPA positive, demonstrating that not all ACPA-positive patients can be detected by testing for CCP reactivity. Moreover, a subset of CCP positive RA patients was single-positive for cTNC5 antibodies (5.4%), revealing cTNC5 as a distinct ACPA fine specificity in RA sera and indicating that assaying this ACPA alone would be helpful in diagnosing patients that might otherwise be missed. Combined testing for several specific ACPAs has been shown to increase both diagnostic sensitivity and specificity[0]. Together these data suggest that both the addition of cTNC5 to an assay combining multiple ACPA, as well as analysis of anti-cTNC5 alone, might be beneficial approaches in diagnosing RA.

HLA-DRB1 SE alleles are associated with ACPA-positive RA[41]. We found a strong association of anti-cTNC5 antibodies with SE positivity, as has been described for antibodies to other citrullinated antigens, like CEP-1 and cVIM[14,15]. cTNC5 antibodies mainly associated with HLA-DRB1 subtypes DRB1*04 as described for other ACPA[15,39]. HLA-DR13 alleles protect against ACPA positive RA[42], and we show here that it is also protective against cTNC5 positive RA. However, we did not observe a statistical significant association of PTPN22, another genetic risk factor for RA[43], with anti-cTNC5 positive RA. Smoking is a well-established environmental risk factor for ACPA positive RA[34,44] and here we describe a positive association of cTNC5 positive RA with smoking in the EIRA cohort, similarly as it has been described for antibodies against CEP-1 and cVIM[14,15]. Smoking-induced inflammation, in the context of chronic obstructive pulmonary disease (COPD), is associated with enhanced citrullination and may contribute to the generation of ACPA[45,46]. Interestingly, high tenascin-C expression was detected in lungs of COPD patients compared to non-smokers[4-8]. Another risk factor for RA, is periodontitis[49]. Porphyromonas gingivalis is a major periodontal pathogen and possesses a unique bacterial PAD enzyme (PPAD) which citrullinates both bacterial and endogenous host peptides[50,51]. Tenascin-C is also expressed in periodontal tissue, and tenascin-C fragments were detected in gingival crevicular fluid of periodontitis patients[52]. Our results and these studies therefore reveal potential mechanisms for the generation of antigenic cTNC peptides in RA.

ACPAs are produced locally within the RA joint and may contribute directly to disease pathogenesis[53]. For example immune complexes containing citrullinated fibrinogen stimulate cytokine synthesis in macrophages via activation of Fcγ-receptor and TLR4 and, due to the homology of fibrinogen and the FBG domain of tenascin-C, it is conceivable that immune complexes containing citrullinated tenascin-C may contribute to disease pathogenesis through a similar mechanism. Furthermore, citrullinated proteins themselves can be pathogenic, as described for citrullinated fibrinogen [54]. It will be interesting to see if ACPA for cTNC5 bind to citrullinated tenascin-C found within the RA joint and trigger cytokine formation in the form of immune complexes, or whether citrullination of the FBG domain enhances its activation of TLR4[26]. The citrullinated FBG peptide previously detected in RA synovial fluid[31] comprised the sequence we found in cTNC1. However, further citrullinated sites and ACPA epitopes are likely to be found in other domains of tenascin-C. For example the fibronectin type-III like repeats that share sequence homology with fibronectin, a molecule also found in synovial fluid and which is targeted by the autoimmune response in RA[55].

The CCP positive subset of RA patients is linked with a more severe disease development and worse prognosis[5,7,8]. However, no association of specific ACPAs with clinical parameters has been described so far[56]. Here, we found that cTNC5 antibodies do not correlate with a number of clinical parameters, however, there was a significant association of cTNC5 antibodies with disease activity (DAS28-CRP), suggesting that cTNC5 antibodies may be useful tool for predicting clinical outcome.

In conclusion, we describe an immunodominant peptide from tenascin-C, which is distinct from the other major antigenic citrullinated peptides described to date, and superior in terms of diagnostic sensitivity and specificity when used as an antigen in ELISA. Furthermore, previous demonstrations of the pro-inflammatory effects of tenascin-C and its detection at site of inflammation suggest that immune responses to the FBG domain may be important in the aetiology and pathogenesis of RA.

### Abbreviations

American College of Rheumatology (ACR)
anti-citrullinated protein antibody (ACPA)
anti-modified citrulline (AMC)
arbitrary units (AU)
chronic obstructive pulmonary disease (COPD)
citrullinated α-enolase peptide 1 (CEP-1)
citrullinated fibrinogen (cFib)
citrullinated vimentin (cVim)
cyclic-citrullinated peptide (CCP)
extracellular matrix (ECM)
Epidemiological Investigation of Rheumatoid Arthritis (EIRA)
fibrinogen-like globe (FBG)
melting temperature (Tm)
odds ratio (OR)
osteoarthritis (OA)
peptidyl arginine deiminases (PAD)
Porphyromonas gingivalis PAD (PPAD)
rheumatoid arthritis (RA)
shared epitope (SE)
toll-like receptor 4 (TLR4)

TABLE 1
Sequences of FBG peptides used for ELISAs.

| FBG peptides | Sequence |
|---|---|
| rTNC1 (aa 2026-2040) | CVFLRRKNG-R-ENFYQNWC |
| cTNC1 | CVFLRRKNG-cit-ENFYQNWC |
| rTNC2 (aa 2042-2058) | CAYAAGFGD-R-REEFWLGLC |
| cTNC2 | CAYAAGFGD-cit-REEFWLGLC |
| rTNC3 (aa 2091-2106) | CFSVGDAKT-R-YKLKVEGYC |
| cTNC3 | CFSVGDAKT-cit-YKLKVEGYC |
| rTNC4 (aa 2141-2157) | CKGAFWY-R-NCH-R-VNLMGRC |
| cTNC4 | CKGAFWY-cit-NCH-cit-VNLMGRC |
| rTNC5 (aa 2183-2200) | CEMKL-R-PSNF-R-NLEG-R-R-KRC |
| cTNC5 | CEMKL-cit-PSNF-cit-NLEG- |
| | cit-cit-KRC |

TABLE 2
Fibrinogen peptides homologous
to citrullinated FBG peptides.

| Fibrinogen | |
|---|---|
| peptides | Sequence |
| cFIBβ²⁸¹⁻²⁹⁶ | CVIQN-cit-QDGSVDFG-cit-KWC |
| cFIBβ⁴⁷⁴⁻⁴⁹¹ | CWYSMRKMSMKI-cit-PFFPQQC |
| cFIBγ⁴⁰⁹⁻⁴²⁶ | CTMKIIPFN-cit-LTIGEGQQHC |

TABLE 3
Association between smoking, any SE, PTPN22
and different RA subgroups (in EIRA).

| | |
|---|---|
| Exposure | p-value |

| Subgroup | Never smokers | Ever smokers | OR (95% CI) | (+/+ vs +/−) |
|---|---|---|---|---|
| Controls | 2114(43.46) | 2750(56.54) | 1.0 ref. | |

CCP2/cTNC5

| −/− | 85(41.87) | 118(58.13) | 0.86 | (0.64-1.17) | |
|---|---|---|---|---|---|
| −/+ | 12(30.00) | 28(70.00) | 1.45 | (0.72-2.93) | |
| +/− | 201(33.84) | 393(66.16) | 1.26 | (1.03-1.53) | |
| +/+ | 309(27.01) | 835(72.99) | 1.65 | (1.41-1.93) | 0.0049 |
| | None SE | Any SE | | | |

| | | | |
|---|---|---|---|
| Controls | 959(49.87) | 964(50.13) | 1.0 ref. |

CCP2/cTNC5

| −/− | 94(46.31) | 108(53.20) | 1.15 | (0.85-1.56) | |
|---|---|---|---|---|---|
| −/+ | 15(35.71) | 27(64.29) | 1.75 | (0.91-3.34) | |
| +/− | 218(36.64) | 368(61.85) | 1.68 | (1.37-2.06) | |
| +/+ | 191(16.68) | 941(82.18) | 4.98 | (4.11-6.04) | <0.0001 |
| | None PTPN22 | Any PTPN22 | | | |

| | | | |
|---|---|---|---|
| Controls | 1533(79.18) | 403(20.82) | 1.0 ref. |

CCP2/cTNC5

| | | | | | |
|---|---|---|---|---|---|
| −/− | 126(62.07) | 50(24.63) | 1.65 | (1.13-2.41) | |
| −/+ | 24(57.14) | 12(28.57) | 2.15 | (1.03-4.49) | |
| +/− | 371(62.35) | 136(22.86) | 1.44 | (1.12-1.86) | |
| +/+ | 682(59.56) | 314(27.42) | 1.77 | (1.44-2.18) | 0.08 |

TABLE 4
Associations between DR*01, *04 and *10 and different RA
subgroups with the combination of presence or absence of
CCP and cTNC5. OR were adjusted for age, gender, residential
area, smoking, alcohol consumption and other SE.

| | | | | p-value |
|---|---|---|---|---|
| | Exposure | | | (+/+ |
| Subgroup | None | Any | OR**(95% CI) | vs +/−) |

DR*01

| | | | |
|---|---|---|---|
| Controls | 375(59.43) | 256(40.57) | 1.0ref. |

CCP2/cTNC5

| | | | | |
|---|---|---|---|---|
| −/− | 62(30.54) | 45(22.17) | 0.91(0.40-2.11) | |
| −/+ | 17(40.48) | 10(23.81) | NA | |
| +/− | 238(40.00) | 129(21.68) | 1.00(0.62-1.63) | |
| +/+ | 627(54.76) | 310(27.07) | 1.81(1.26-2.60) | 0.01 |

DR*04

| | | | |
|---|---|---|---|
| Controls | 220(34.87) | 411(65.13) | 1.0ref. |

CCP2/cTNC5

| | | | | |
|---|---|---|---|---|
| −/− | 41(20.20) | 66(32.51) | 0.77(0.33-1.84) | |
| −/+ | 13(30.95) | 14(33.33) | NA | |
| +/− | 110(18.49) | 257(43.19) | 1.29(0.78-2.15) | |
| +/+ | 192(16.77) | 745(65.07) | 3.46(2.35-5.11) | <0.0001 |

DR*10

| | | | |
|---|---|---|---|
| Controls | 609(96.51) | 22(3.49) | 1.0ref. |

CCP2/cTNC5

| | | | | |
|---|---|---|---|---|
| −/− | 102(50.25) | 5(2.46) | 1.18(0.36-3.87) | |
| −/+ | 24(57.14) | 3(7.14) | NA | |
| +/− | 351(58.99) | 16(2.69) | 1.53(0.71-3.29) | |
| +/+ | 898(78.43) | 39(3.41) | 2.11(1.15-3.88) | 0.13 |

NA—not analysed.

TABLE 5
Association between DR13 and different RA subgroups
with the combination of presence or absence of CCP and
cTNC5. OR were adjusted for age, gender, residential
area, smoking, alcohol consumption and any SE.

| | Exposure | | | |
|---|---|---|---|---|
| Subgroup | No DR13 | Any DR13 | OR (95% CI) | p-value |
| Controls | 503(79.71) | 128(20.29) | 1.0ref. | |

CCP2/cTNC5

| | | | | |
|---|---|---|---|---|
| −/− | 86(42.36) | 21(10.34) | 0.84(0.49-1.46) | |
| −/+ | 25(59.52) | 2(4.76) | 0.31(0.07-1.34) | (−/+ |
| | | | | vs −/−) |
| | | | | 0.16 |
| +/− | 315(52.94) | 52(8.74) | 0.67(0.47-0.97) | |
| +/+ | 867(75.72) | 70(6.11) | 0.30(0.22-0.42) | (+/+ vs |
| | | | | +/−) |
| | | | | 0.0003 |

TABLE 6
Association of cTNC5 antibodies with RA disease
characteristics. Numbers in brackets are s.d.

| | | cTNC5 | | cTNC5 | | |
|---|---|---|---|---|---|---|
| | Total | positive | | negative | | |
| Characteristic | (N = 287) | (N = 145) | | (N = 142) | | p-value |
| Age, years | | 60 | (12) | 58 | (11) | 0.252 |
| Male gender | | 68 | | 58 | | 0.084 |
| Dis. duration, years | | 13 | (10) | 12 | (9) | 0.189 |
| Swollen joints | | 3.9 | (4.7) | 3.2 | (3.8) | 0.212 |
| Tender joints | | 3.4 | (4.8) | 2.9 | (4.4) | 0.366 |
| Patient global (0-10) | | 4.5 | (2.8) | 3.9 | (2.5) | 0.067 |
| DAS-28-CRP | | 4.1 | (1.4) | 3.8 | (1.3) | 0.044 |
| Sharp score | | 21 | (23) | 17 | (22) | 0.061 |
| Erosion score | | 5 | (8) | 4 | (8) | 0.632 |
| Anti-CCP, U/ml | | 187 | (123) | 101 | (117) | <0.001 |

TABLE 7

| Peptide | Protein | Amino acids | Amino Acid sequence |
|---|---|---|---|
| CEP-1/Eno5-21 | α-Enolase | 5-21 | CKIHA(cit)EIFDS(cit)GNPTVEC (cyclic) |
| Vim60-75 | Vimentin | 60-75 | VYAT(cit)SSAV(cit)L(cit)SSVP |
| Vim2-17 | Vimentin | 2-17 | ST(cit)SVSSSSY(cit)(cit)MFGG |
| CCP1/Fil307-324 | Filaggrin | 307-324 | SHQEST(cit)GRSRGRSGRSGS (cyclic) |
| Fibβ36-52 | Fibrinogen β-chain | 36-52 | NEEGFFSA(cit)GHRPLDKK |
| Fibβ563-583 | Fibrinogen β-chain | 563-583 | HHPGIAEFPS(cit)GKSSSYSKQF |
| Fibβ580-600 | Fibrinogen β-chain | 580-600 | SKQFTSSTSYN(cit)GQSTFESKS |
| Fibβ62-81a^{a} | Fibrinogen β-chain | 62-81 | APPPISGGGY(cit)ARPAKAAAT |
| Fibβ62-81b^{b} | Fibrinogen β-chain | 62-81 | APPPISGGGYRA(cit)PAKAAAT |
| Fibβ60-74 | Fibrinogen β-chain | 60-74 | (cit)PAPPPISGGGY(cit)A(cit) |
| Fibα621-635 | Fibrinogen α-chain | 621-635 | (cit)GHAKS(cit)PV(cit)GIHTS |
| citCI/CII359-369 | Collagen type II | 359-369 | (GPO)S-GA(cit)GLTG(cit)PGDA(GPO)2-GKKYG |

Sequences
TNC sequence- Accession No. P24821
>sp|P24821|TENA_HUMAN Tenascin OS = Homo sapiens
GN = TNC PE = 1 SV = 3
SEQ ID NO: 1
MGAMTQLLAGVFLAFLALA1EGGVLKKVIRHKRQSGVNATLPEENQPVV
FNHVYNIKLPVGSQCSVDLESASGEKDLAPPSEPSESFQEHTVDGENQI
VFTHRINIPRRACGCAAAPDVKELLSRLEELENLVSSLREQCTAGAGCC
LQPATGRLDTRPFCSGRGNFSTEGCGCVCEPGWKGPNCSEPECPGNCHL
RGRCIDGQCICDDGFTGEDCSQLACPSDCNDQGKCVNGVCICFEGYAGA
DCSREICPVPCSEEHGTCVDGLCVCHDGFAGDDCNKPLCLNNCYNRGRC
VENECVCDEGFTGEDCSELICPNDCFDRGRCINGTCYCEEGFTGEDCGK
PTCPHACHTQGRCEEGQCVCDEGFAGVDCSEKRCPADCHNRGRCVDGRC
ECDDGFTGADCGELKCPNGCSGHGRCVNGQCVCDEGYTGEDCSQLRCPN
DCHSRGRCVEGKCVCEQGFKGYDCSDMSCPNDCHQHGRCVNGMCVCDDG
YTGEDCRDRQCPRDCSNRGLCVDGQCVCEDGFTGPDCAELSCPNDCHGQ
GRCVNGQCVCHEGFMGKDCKEQRCPSDCHGQGRCVDGQCICHEGFTGLD
CGQHSCPSDCNNLGQCVSGRCICNEGYSGEDCSEVSPPKDLVVTEVTEE
TVNLAWDNEMRVTEYLVVYTPTHEGGLEMQFRVPGDQTSTIIQELEPGV
EYFIRVFAILENKKSIPVSARVATYLPAPEGLKFKSIKETSVEVEWDPL
DIAFETWEIIFRNMNKEDEGEITKSLRRPETSYRQTGLAPGQEYEISLH
IVKNNTRGPGLKRVTTTRLDAPSQIEVKDVTDTTALITWFKPLAEIDGI
ELTYGIKDVPGDRTTIDLTEDENQYSIGNLKPDTEYEVSLISRRGDMSS
NPAKETFTTGLDAPRNLRRVSQTDNSITLEWRNGKAAIDSYRIKYAPIS
GGDHAEVDVPKSQQATTKTTLTGLRPGTEYGIGVSAVKEDKESNPATIN
AATELDTPKDLQVSETAETSLTLLWKTPLAKFDRYRLNYSLPTGQWVGV
QLPRNTTSYVLRGLEPGQEYNVLLTAEKGRHKSKPARVKASTEQAPELE
NLTVTEVGWDGLRLNWTAADQAYEHFIIQVQEANKVEAARNLTVPGSLR
AVDIPGLKAATPYTVSIYGVIQGYRTPVLSAEASTGETPNLGEVVVAEV
GWDALKLNWTAPEGAYEYFFIQVQEADTVEAAQNLTVPGGLRSTDLPGL
KAATHYTITIRGVTQDFSTTPLSVEVLTEEVPDMGNLTVTEVSWDALRL
NWTTPDGTYDQFTIQVQEADQVEEAHNLTVPGSLRSMEIPGLRAGTPYT
VTLHGEVRGHSTRPLAVEVVTEDLPQLGDLAVSEVGWDGLRLNWTAADN
AYEHFVIQVQEVNKVEAAQNLTLPGSLRAVDIPGLEAATPYRVSIYGVI
RGYRTPVLSAEASTAKEPEIGNLNVSDITPESFNLSWMATDGIFETFTI
EIIDSNRLLETVEYNISGAERTAHISGLPPSTDFIVYLSGLAPSIRTKT
ISATATIEALPLLENLTISDINPYGFTVSWMASENAFDSFLVTVVDSGK
LLDPQEFTLSGTQRKLELRGLITGIGYEVMVSGFTQGHQTKPLRAEIVT
EAEPEVDNLLVSDATPDGFRLSWTADEGVFDNFVLKIRDTKKQSEPLEI
TLLAPERTRDITGLREATEYEIELYGISKGRRSQTVSAIATTAMGSPKE
VIFSDITENSATVSWRAPTAQVESFRITYVPITGGTPSMVTVDGTKTQT
RLVKLIPGVEYLVSIIAMKGFEESEPVSGSFTTALDGPSGLVTANITDS
EALARWQPAIATVDSYVISYTGEKVPEITRTVSGNTVEYALTDLEPATE
YTLRIFAEKGPQKSSTITAKFTTDLDSPRDLTATEVQSETALLTWRPPR
ASVTGYLLVYESVDGTVKEVIVGPDTTSYSLADLSPSTHYTAKIQALNG
PLRSNMIQTIFTTIGLLYPFPKDCSQAMLNGDTTSGLYTIYLNGDKAEA
LEVFCDMTSDGGGWIVFLRRKNGRENFYQNWKAYAAGFGDRREEFWLGL
DNLNKITAQGQYELRVDLRDHGETAFAVYDKFSVGDAKTRYKLKVEGYS
GTAGDSMAYHNGRSFSTFDKDTDSAITNCALSYKGAFWYRNCHRVNLMG
RYGDNNHSQGVNWFHWKGHEHSIQFAEMKLRPSNFRNLEGRRKRA

Detection of Antibodies to Citrullinated Tenascin-C in Patients with Early Synovitis is Associated with the Development of Rheumatoid Arthritis

Early treatment of rheumatoid arthritis (RA) results in more effective disease suppression and can be key to a successful patient response. However, not all patients with early synovitis develop RA; for example, in some, synovial inflammation resolves spontaneously. The factors that drive RA development remain unclear and clinical tools to predict RA development are imperfect.

Tenascin-C is a pro-inflammatory matrix molecule that is absent from healthy joints but highly expressed in the joints of RA patients. We identified an immunodominant peptide in citrullinated tenascin-C, cTNC5, antibodies against which are detected in around half of RA patients, and can be found years before disease onset in some patients. Here, we sought to determine if anti-cTNC5 antibodies can discriminate amongst patients with early synovial inflammation those who develop RA and those with other outcomes.

Sera from 263 patients in the Birmingham early arthritis cohort were analysed. Patients were DMARD naïve with clinically apparent synovitis of ≥1 joint and with inflammatory joint symptoms of ≤3 months duration. At 18 month follow-up patients were assigned to the following outcome categories: RA according to ACR 2010 criteria (Arnett F C, et al. Arthritis and rheumatism. 1988; 31(3):315-24.) (RA, n=101), persistent non-RA arthritis (PNRA, n=66) and resolving arthritis (no clinically apparent joint swelling, no DMARD/steroid use in the previous 3 months, n=96). Demographic and clinical parameters were recorded, and RA patients divided into anti-CCP antibody positive and negative subsets (Raza K, et al. The Journal of rheumatology. 2005; 32(2):231-8; and Raza K, et al. Arthritis research & therapy. 2005; 7(4):R784-95). Antibodies recognizing cTNC5 or a non-citrullinated peptide (rTNC5) were analysed by ELISA as described (Schwenzer A, et al. Annals of the rheumatic diseases. 2015).

Anti-cTNC5 antibodies were found in 40.6% of people with early synovitis that went on to develop RA, and were significantly more prevalent in anti-CCP antibody +ve compared to anti-CCP antibody −ve RA patients (81.3 vs. 3.8%, p<0.0001). Anti-cTNC5 antibodies were detected in a low proportion of people who developed PNRA (6.1%), or whose disease resolved (3.1%). No significant antibody response to rTNC5 was detected (p=0.527) (Table 8, FIG. 10). Anti-cTNC5 levels were higher in anti-CCP antibody +ve RA patients (193.1±449.8 AU) compared to patients with in anti-CCP antibody −ve RA (3.56±3.30 AU), PNRA (19.42±122.7 AU) and resolving arthritis (6.60±28.02 AU) ANOVA p<0.0001).

Whilst anti-cTNC5 was not better at predicting the development of RA than anti-CCP antibody (specificity; sensitivity: 40.6%; 95.7% (cTNC5), 47.5%; 98.8% (CCP), anti-cTNC5 did detect a subset of patients that developed RA who were not anti-CCP antibody positive (3.8%). Anti-cTNC5 antibody positive RA patients were more frequently anti-CCP antibody and RF positive than anti-cTNC5 antibody negative patients (Table 9).

In addition to anti-cTNC5 predicting the development of RA, cTNC5 positive individuals had significantly higher CRP and ESR levels, higher disease activity scores, and higher tender and swollen joint counts than cTNC5 negative individuals (Table 10); there has previously been reported no difference in clinical phenotype between CCP+ve or CCP−ve RA patients (Cader M Z, et al. BMC musculoskeletal disorders. 2010; 11:187).

Together these data reveal that detection of anti-cTNC5 antibodies in the sera of patients with early synovitis is associated with the development of RA, and particularly with high levels of disease activity. This study therefore highlights a potential role for citrullinated tenascin-C in the biological pathways underlying the differentiation of early synovitis towards RA and away from disease resolution.

TABLE 8
Demographic, clinical and laboratory characteristics of patients in each outcome group.

| | Anti-CCP | | Anti-CCP | | Persistent | | Resolving | | |
|---|---|---|---|---|---|---|---|---|---|
| | negative RA | | positive RA | | non-RA | | arthritis | | |
| | (n = 53) | | (n = 48) | | (n = 66) | | (n = 96) | | P-Value |
| Female, n (%) | 27 | (50.9) | 31 | (64.6) | 37 | (56.1) | 46 | (47.9) | 0.274 |
| Age (years) | 55.6 ± 15.7 | | 55.5 ± 14.4 | | 52.1 ± 18.9 | | 45.9 ± 16.8 | | <0.0001 |
| Disease duration (days) | 52.4 ± 21.4 | | 55.3 ± 21.7 | | 56.4 ± 21.5 | | 45.3 ± 20.8 | | 0.005 |
| CRP (mg/dl) | 10 | (0-39) | 17.5 | (6-43.8) | 20.5 | (7.5-35.3) | 7 | (0-17) | <0.0001 |
| ESR (mm/hour) | 18 | (11.5-44.5) | 27.5 | (18.3-51.3) | 21.5 | (7.8-45.8) | 12.5 | (5-27) | <0.0001 |
| DAS28 (CRP) | 4.4 ± 1.4 | | 4.4 ± 1.4 | | 3.6 ± 1.2 | | 2.8 ± 1.3 | | <0.0001 |
| DAS28 (ESR) Smoking n (%) | 4.6 ± 1.5 | | 4.7 ± 1.6 | | 3.6 ± 1.8 | | 2.9 ± 1.5 | | <0.001 |
| Ever smoker | 28/49 | (57-1) | 27/47 | (57.4) | 26/64 | (40.6) | 35/89 | (39.3) | 0.07 |
| Never smoker | 21/49 | (42.9) | 20/47 | (42.6) | 38/64 | (59.4) | 54/89 | (60.7) | |
| Anti-CCP positive, n (%) | 0 | (0) | 48 | (100) | 1 | (1.5) | 1 | (1.0) | <0.0001 |
| cTNC5 positive, n (%) | 2 | (3.8) | 39 | (81.3) | 4 | (6.1) | 3 | (3.1) | <0.0001 |
| rTNC5 positive, n (%) | 1 | (1.9) | 1 | (2.1) | 3 | (4.5) | 1 | (1.0) | 0.527 |

Data are shown as number (percentage), mean +/− SD, or median (IQR) as appropriate.
CCP, cyclic citrullinated peptide; CRP, C reactive protein; DAS, disease activity score; ESR, erythrocyte sedimentation rate; RA, rhematoid arthritits; RF, rheumatoid factor; v cTNC, citrullinated tenascin-C.

TABLE 9
Characteristics of RA patients with
and without anti-cTNC5 antibodies.

| | | |
|---|---|---|
| Anti-cTNC5 | Anti-cTNC5 | |
| negative RA | positive RA | |
| (n = 60) | (n = 41) | P value |

| | | | | | |
|---|---|---|---|---|---|
| Female, n (%) | 33 | (55) | 25 | (60.1) | 0.682 |

| | | | |
|---|---|---|---|
| Age (years) | 55.2 ± 16.1 | 56.1 ± 13.3 | 0.785 |
| Symptom | 52.3 ± 21.5 | 56 ± 21.5 | 0.400 |
| duration | | | |
| (days) | | | |

| | | | | | |
|---|---|---|---|---|---|
| CRP (mg/dl) | 10.5 | (0-43) | 18 | (6-39) | 0.062 |
| ESR (mm/hour) | 18 | (11-45) | 25 | (19-46) | 0.372 |

| | | | |
|---|---|---|---|
| DAS28 (CRP) | 4.26 ± 1.4 | 4.55 ± 1.4 | 0.320 |
| DAS28 (ESR) | 4.51 ± 1.5 | 4.82 ± 1.6 | 0.320 |
| 28 TJC | 7.22 ± 6.5 | 9.1 ± 10.4 | 0.267 |
| 28 SJC | 7.6 ± 7.2 | 6.9 ± 5.5 | 0.595 |

| | | | | | |
|---|---|---|---|---|---|
| Smoking, n (%) | | | | | |
| Ever smoker | 34/56 | (60.7) | 21/40 | (52.5) | 0.682 |
| Never smoker | 22/56 | (39.3) | 19/40 | (47.5) | 0.374 |
| Anti-CCP | 9 | (15) | 39 | (95.1) | <0.0001 |
| positive, n (%) | | | | | |

Data are shown as number (percentage), mean +/− SD, or median (IQR) as appropriate. Comparisons have been performed with χ2, T test and Mann Whiteny U test for categorical, parametric continuous and non-parametric continuous data, respectively.
cTNC, citrullinated tenascin-C; CRP, C reactive protein; ESR, erythrocyte sedimentation rate; DAS, disease activity score; TJC, tender joint count; SJC, swollen joint count; CCP, cyclic citrullinated peptide.

TABLE 10
Characteristics of early synovitis patients
with and without anti-cTNC5 antibodies.

| | cTNC5 | | cTNC5 | | |
|---|---|---|---|---|---|
| | negative | | positive | | P value |
| Female, n (%) | 112 | (52.1) | 29 | (60.4) | 0.188 |
| Age (years) | 50.4 ± 17.7 | | 54.6 ± 13.7 | | 0.123 |
| Disease | 50.6 ± 21.7 | | 54.9 ± 21.6 | | 0.124 |
| duration | | | | | |
| (days) | | | | | |
| CRP (mg/dl) | 10 | (0-28) | 18 | (6-43.8) | 0.021 |
| ESR (mm/hour) | 17 | (7-38) | 25 | (18.3-44.8) | 0.002 |
| DAS28 (CRP) | 3.4 ± 1.4 | | 4.4 ± 1.4 | | <0.0001 |
| DAS28 (ESR) | 3.6 ± 1.7 | | 4.6 ± 1.7 | | <0.0001 |
| 28 TJC | 4.2 ± 5.5 | | 8.1 ± 9.9 | | <0.0001 |
| 28 SJC | 3.9 ± 5.3 | | 6.5 ± 5.4 | | 0.002 |
| Smoking, n (%) | | | | | |
| Ever smoker | 90/202 | (44.5) | 21/47 | (44.7) | 0.343 |
| Never smoker | 112/202 | (55.5) | 26/47 | (55.3) | 0.374 |
| Anti-CCP | 10 | (4.7) | 40 | (83.3) | <0.0001 |
| positive, n (%) | | | | | |

Data are shown as number (percentage), mean +/− SD, or median (IQR) as appropriate. cTNC, citrullinated tenascin-C; CRP, C reactive protein; ESR, erythrocyte sedimentation rate; DAS, disease activity score; TJC, tender joint count; SJC, swollen joint count; CCP, cyclic citrullinated peptide.

### REFERENCES

1. Kinloch A, Lundberg K, Wait R, et al. Synovial fluid is a site of citrullination of autoantigens in inflammatory arthritis. Arthritis Rheum. 2008; 58:2287-95.
2. Vossenaar E R, Smeets T J, Kraan M C, et al. The presence of citrullinated proteins is not specific for rheumatoid synovial tissue. Arthritis Rheum. 2004; 50:3485-94.
3. Girbal-Neuhauser E, Durieux J J, Arnaud M, et al. The epitopes targeted by the rheumatoid arthritis-associated antifilaggrin autoantibodies are posttranslationally generated on various sites of (pro)filaggrin by deimination of arginine residues. J Immunol. 1999; 162:585-94.
4. Schellekens G A, de Jong B A, van den Hoogen F H, et al. Citrulline is an essential constituent of antigenic determinants recognized by rheumatoid arthritis-specific autoantibodies. J Clin Invest. 1998; 101:273-81.
5. Kroot E J, de Jong B A, van Leeuwen M A, et al. The prognostic value of anti-cyclic citrullinated peptide antibody in patients with recent-onset rheumatoid arthritis. Arthritis Rheum. 2000; 43:1831-5.
6. Nielen M M, van Schaardenburg D, Reesink H W, et al. Specific autoantibodies precede the symptoms of rheumatoid arthritis: a study of serial measurements in blood donors. Arthritis Rheum. 2004; 50:380-6.
7. Rantapaa-Dahlqvist S, de Jong B A, Berglin E, et al. Antibodies against cyclic citrullinated peptide and IgA rheumatoid factor predict the development of rheumatoid arthritis. Arthritis Rheum. 2003; 48:2741-9.
8. Vencovsky J, Machacek S, Sedova L, et al. Autoantibodies can be prognostic markers of an erosive disease in early rheumatoid arthritis. Ann Rheum Dis. 2003; 62:427-30.
10. Wegner N, Lundberg K, Kinloch A, et al. Autoimmunity to specific citrullinated proteins gives the first clues to the etiology of rheumatoid arthritis. Immunol Rev. 2010; 233:34-54.
13. Lundberg K, Kinloch A, Fisher B A, et al. Antibodies to citrullinated alpha-enolase peptide 1 are specific for rheumatoid arthritis and cross-react with bacterial enolase. Arthritis Rheum. 2008; 58:3009-19.
14. Lundberg K, Bengtsson C, Kharlamova N, et al. Genetic and environmental determinants for disease risk in subsets of rheumatoid arthritis defined by the anticitrullinated protein/peptide antibody fine specificity profile. Ann Rheum Dis. 2013; 72:652-8.
19. Midwood K S, Orend G. The role of tenascin-C in tissue injury and tumorigenesis. J Cell Commun Signal. 2009; 3:287-310.
20. Udalova I A, Ruhmann M, Thomson S J, et al. Expression and immune function of tenascin-C. Crit Rev Immunol. 2011; 31:115-45.
21. Chevalier X, Groult N, Larget-Piet B, et al. Tenascin distribution in articular cartilage from normal subjects and from patients with osteoarthritis and rheumatoid arthritis. Arthritis Rheum. 1994; 37:1013-22.
22. Goh F G, Piccinini A M, Krausgruber T, et al. Transcriptional regulation of the endogenous danger signal tenascin-C: a novel autocrine loop in inflammation. J Immunol. 2010; 184:2655-62.
23. Hasegawa M, Nakoshi Y, Muraki M, et al. Expression of large tenascin-C splice variants in synovial fluid of patients with rheumatoid arthritis. J Orthop Res. 2007; 25:563-8.
24. Salter D M. Tenascin is increased in cartilage and synovium from arthritic knees. Br J Rheumatol. 1993; 32:780-6.
25. Page T H, Charles P J, Piccinini A M, et al. Raised circulating tenascin-C in rheumatoid arthritis. Arthritis Res Ther. 2012; 14:R260.
26. Midwood K, Sacre S, Piccinini A M, et al. Tenascin-C is an endogenous activator of Toll-like receptor 4 that is essential for maintaining inflammation in arthritic joint disease. Nat Med. 2009; 15:774-80.
30. Monach P A, Hueber W, Kessler B, et al. A broad screen for targets of immune complexes decorating arthritic joints highlights deposition of nucleosomes in rheumatoid arthritis. Proc Natl Acad Sci USA. 2009; 106:15867-72.
31. Tutturen A E, Fleckenstein B, de Souza G A. Assessing the citrullinome in rheumatoid arthritis synovial fluid with and without enrichment of citrullinated peptides. J Proteome Res. 2014; 13:2867-73.
32. Arnett F C, Edworthy S M, Bloch D A, et al. The American Rheumatism Association 1987 revised criteria for the classification of rheumatoid arthritis. Arthritis Rheum. 1988; 31:315-24.
33. Cartwright A J, Quirke A-M, de Pablo P, et al. Smoking Affects Immune Response to Citrullinated Autoantigens Before Clinical Onset of Rheumatoid Arthritis in a Southern European Cohort [abstract]. Rheumatology (Oxford). 2014; 53:i36.
34. Stolt P, Bengtsson C, Nordmark B, et al. Quantification of the influence of cigarette smoking on rheumatoid arthritis: results from a population based case-control study, using incident cases. Ann Rheum Dis. 2003; 62:835-41.
35. Mikuls T R, Payne J B, Yu F, et al. Periodontitis and Porphyromonas gingivalis in patients with rheumatoid arthritis. Arthritis Rheumatol. 2014; 66:1090-100.
36. Xu D, Suenaga N, Edelmann M J, et al. Novel MMP-9 substrates in cancer cells revealed by a label-free quantitative proteomics approach. Mol Cell Proteomics. 2008; 7:2215-28.
37. Assohou-Luty C, Raijmakers R, Benckhuijsen W E, et al. The human peptidylarginine deiminases type 2 and type 4 have distinct substrate specificities. Biochim Biophys Acta. 2014; 1844:829-36.
38. van Beers J J, Raijmakers R, Alexander L E, et al. Mapping of citrullinated fibrinogen B-cell epitopes in rheumatoid arthritis by imaging surface plasmon resonance. Arthritis Res Ther. 2010; 12:R219.
39. Snir O, Widhe M, von Spee C, et al. Multiple antibody reactivities to citrullinated antigens in sera from patients with rheumatoid arthritis: association with HLA-DRB1 alleles. Ann Rheum Dis. 2009; 68:736-43.
40. Wagner C A, Sokolove J, Lahey L J, et al. Identification of anticitrullinated protein antibody reactivities in a subset of anti-CCP-negative rheumatoid arthritis: association with cigarette smoking and HLA-DRB1 ‘shared epitope’ alleles. Ann Rheum Dis. 2015; 74:579-86.
41. Huizinga T W, Amos C I, van der Helm-van Mil A H, et al. Refining the complex rheumatoid arthritis phenotype based on specificity of the HLA-DRB1 shared epitope for antibodies to citrullinated proteins. Arthritis Rheum. 2005; 52:3433-8.
42. van der Woude D, Lie B A, Lundstrom E, et al. Protection against anti-citrullinated protein antibody-positive rheumatoid arthritis is predominantly associated with HLA-DRB1*1301: A meta-analysis of HLA-DRB1 associations with anti-citrullinated protein antibody-positive and anti-citrullinated protein antibody-negative rheumatoid arthritis in four European populations. Arthritis Rheum. 2010; 62:1236-45.
43. Plenge R M, Padyukov L, Remmers E F, et al. Replication of putative candidate-gene associations with rheumatoid arthritis in >4,000 samples from North America and Sweden: association of susceptibility with PTPN22, CTLA4, and PADI4. Am J Hum Genet. 2005; 77:1044-60.
44. Silman A J, Newman J, MacGregor A J. Cigarette smoking increases the risk of rheumatoid arthritis. Results from a nationwide study of disease-discordant twins. Arthritis Rheum. 1996; 39:732-5.
45. Klareskog L, Catrina A I. Autoimmunity: lungs and citrullination. Nat Rev Rheumatol. 2015; 11:261-2.
46. Lugli E B, Correia R E, Fischer R, et al. Expression of citrulline and homocitrulline residues in the lungs of non-smokers and smokers: implications for autoimmunity in rheumatoid arthritis. Arthritis Res Ther. 2015; 17:9.
47. Annoni R, Lancas T, Yukimatsu Tanigawa R, et al. Extracellular matrix composition in COPD. Eur Respir J. 2012; 40:1362-73.
48. Lofdahl M, Kaarteenaho R, Lappi-Blanco E, et al. Tenascin-C and alpha-smooth muscle actin positive cells are increased in the large airways in patients with COPD. Respir Res. 2011; 12:48.
49. Rosenstein E D, Greenwald R A, Kushner L J, et al. Hypothesis: the humoral immune response to oral bacteria provides a stimulus for the development of rheumatoid arthritis. Inflammation. 2004; 28:311-8.
50. Wegner N, Wait R, Sroka A, et al. Peptidylarginine deiminase from Porphyromonas gingivalis citrullinates human fibrinogen and alpha-enolase: implications for autoimmunity in rheumatoid arthritis. Arthritis Rheum. 2010; 62:2662-72.
51. Montgomery A B, Kopec J, Shrestha L, et al. Crystal structure of Porphyromonas gingivalis peptidylarginine deiminase: implications for autoimmunity in rheumatoid arthritis. Ann Rheum Dis. 2015.
52. Ruggiero S, Cosgarea R, Potempa J, et al. Cleavage of extracellular matrix in periodontitis: gingipains differentially affect cell adhesion activities of fibronectin and tenascin-C. Biochim Biophys Acta. 2013; 1832:517-26.
53. Kuhn K A, Kulik L, Tomooka B, et al. Antibodies against citrullinated proteins enhance tissue injury in experimental autoimmune arthritis. J Clin Invest. 2006; 116:961-73.
54. Sanchez-Pernaute O, Filkova M, Gabucio A, et al. Citrullination enhances the pro-inflammatory response to fibrin in rheumatoid arthritis synovial fibroblasts. Ann Rheum Dis. 2013; 72:1400-6.
55. van Beers J J, Willemze A, Stammen-Vogelzangs J, et al. Anti-citrullinated fibronectin antibodies in rheumatoid arthritis are associated with human leukocyte antigen-DRB1 shared epitope alleles. Arthritis Res Ther. 2012; 14:R35.
56. Fisher B A, Plant D, Brode M, et al. Antibodies to citrullinated alpha-enolase peptide 1 and clinical and radiological outcomes in rheumatoid arthritis. Ann Rheum Dis. 2011; 70:1095-8.
57. Kallberg H, Padyukov L, Plenge R M, et al. Gene-gene and gene-environment interactions involving HLA-DRB1, PTPN22, and smoking in two subsets of rheumatoid arthritis. Am J Hum Genet. 2007; 80:867-75.
58. Padyukov L, Silva C, Stolt P, et al. A gene-environment interaction between smoking and shared epitope genes in HLA-D R provides a high risk of seropositive rheumatoid arthritis. Arthritis Rheum. 2004; 50:3085-92.
59. Fischer R, Kessler B M. Gel-aided sample preparation (GASP)—a simplified method for gel-assisted proteomic sample generation from protein extracts and intact cells. Proteomics. 2015; 15:1224-9.

1. A method of identifying a subject suspected of having or being susceptible to an autoimmune disease, such as rheumatoid arthritis (RA), comprising:
contacting a sample of bodily fluid obtained from the subject with (i) a binding pair member having a binding affinity for citrullinated tenascin (cTNC) or a fragment thereof or (ii) a cTNC peptide;
determining in a sample of bodily fluid obtained from the subject the presence or amount of (i) a citrullinated peptide derived from tenascin or (ii) an anti cTNC antibody;
comparing the presence or amount of (i) the citrullinated peptide derived from tenascin or (ii) the anti cTNC antibody with a pre-defined threshold value; and
assigning a diagnosis of RA or a future likelihood of developing RA when the presence or amount of (i) cTNC or (ii) an antibody against cTNC is detected or exceeds the threshold.

2. The method of claim 1 wherein the binding pair member having an affinity for cTNC is a monoclonal antibody, a polycloncal antibody, or functional binding fragments of each thereof including but not limited to Fab, Fab2, Fv, ScFv, Fc, dAb, Fd, diabodies.

3. The method of claim 1 or 2 wherein the binding pair member is purified from a mammal or is expressed by recombinant DNA technology.

4. The method of claim 1 to 3 wherein the binding pair member has specificity for cTNC in the presence of non-citrullinated TNC.

5. The method of claim 1 wherein the cTNC is cTNC5 as defined in table 1.

6. The method of claim 1 wherein the cTNC has sequence comprising RcitXXXXRcitXXXXRcitRcit, where Rcit is citrulline and X is any amino acid.

7. The method of claim 1 wherein the cTNC is selected from the group comprising RcitXXXXRcitXXXXRcitX1; RcitXXXXRcitXXXXX1Rcit; RcitXXXXX1XXXXRcitRcit; or X1XXXXRcitXXXXRcitRcit; wherein: Rcit is a citrullinated arginine residue; X is any amino acid; and X1 is a non-citrullinated arginine, or any other amino acid.

8. The method of claim 1 wherein the cTNC has sequence comprising RcitPSNFRcitNLEGRcitRcit.

9. The method of claim 1 wherein the cTNC has sequence comprising EHSIQFAEMKLRcitPSNFRcitNLEGRcitRcitKR.

10. The method of claim 1 wherein the cTNC has sequence comprising EHSIQFAEMKLRcitPSNFRcitNLEGRcitRcitKRcit.

11. The method of claim 1 wherein the cTNC has sequence comprising EHSIQFAEMKLRcitPSNFRcitNLEGRcitRcitKRA.

12. The method of claim 1 wherein the cTNC has sequence comprising EHSIQFAEMKLRcitPSNFRcitNLEGRcitRcitKRcitA.

13. The method of any preceding claim wherein the RA is erosive RA.

14. The method of claim 1 to 13 wherein the step of determining the presence or amount of a citrullinated peptide derived from tenascin comprises:
(i) performing a sandwich immunoassay configured with a first binding pair member for cTNC associated with a solid phase and a second binding pair member with a detectable label capable of simultaneous binding to cTNC;
(ii) performing a competitive immunoassay configured with a binding pair member and labelled cTNC analog capable of competing with cTNC for binding to the binding pair member;
(iii) performing a homogeneous immunoassay comprising a binding pair member for cTNC associated with a particle, wherein the presence of cTNC results in formation of aggregates that increase turbidity of the sample;
(iv) detecting changes in the presence or amount of detectable label associated with a binding pair member or labelled cTNC analog in steps (i), (ii) or (iii); and
(v) correlating changes in presence or amount of detectable label with the presence or amount of cTNC in the sample.

15. The method of claim 1 to 13 wherein the step of determining the presence or amount of an anti cTNC antibody comprises:
(i) performing a sandwich immunoassay configured with a first binding pair member for the anti-cTNC antibody associated with a solid phase and a second binding pair member with a detectable label capable of simultaneous binding to the anti-cTNC antibody;
(ii) performing a competitive immunoassay configured with a labelled binding pair member and cTNC immobilised on a solid phase, wherein the anti-cTNC antibody competes with the binding pair member for binding to the immobilised cTNC;
(iii) performing a homogeneous immunoassay comprising a cTNC associated with a particle and a binding pair member for the anti-cTNC antibody, wherein the presence of anti-cTNC antibody results in formation of aggregates that increase the turbidity of the sample;
(iv) detecting changes in the presence or amount of detectable label associated with a binding pair member or cTNC; and
(v) correlating changes in presence or amount of detectable label with the presence or amount of anti-cTNC antibody in the sample.

16. A peptide comprising or consisting of the sequence R_{cit}PSNFR_{cit}NLEGR_{cit}R_{cit}, or a variant thereof, wherein R_{cit} is a citrullinated arginine residue.

17. The peptide according to claim 16, wherein the peptide comprises or consists of the sequence EHSIQFAEMKLR_{cit}PSNFR_{cit}NLEGR_{cit}R_{cit}KR, or a variant peptide thereof, wherein R_{cit} is a citrullinated arginine residue.

18. The peptide according to claim 16, wherein the peptide comprises or consists of the sequence EHSIQFAEMKLR_{cit}PSNFRNLEGR_{cit}R_{cit}KR_{cit}, or a variant peptide thereof, wherein R_{cit} is a citrullinated arginine residue.

19. The peptide according to claim 16, wherein the peptide comprises or consists of the sequence:
EHSIQFAEMKLR_{cit}PSNFR_{cit}NLEGR_{cit}R_{cit}KRA: or
EHSIQFAEMKLR_{cit}PSNFR_{cit}NLEGR_{cit}R_{cit}KR_{cit}A;
or a variant peptide thereof, wherein R_{cit} is a citrullinated arginine residue.

20. The peptide of claim 16, wherein the peptide is a variant peptide comprising or consisting of the sequence R_{cit}XXXXR_{cit}XXXXR_{cit}R_{cit}, wherein R_{cit} is a citrullinated arginine residue, and X is any amino acid.

21. The peptide of claim 16, wherein the peptide is a variant peptide comprising or consisting of any one of the sequences of
R_{cit}XXXXR_{cit}XXXXR_{cit}X₁;
R_{cit}XXXXR_{cit}XXXXX₁R_{cit};
R_{cit}XXXXX₁XXXXR_{cit}R_{cit}; or
X₁XXXXR_{cit}XXXXR_{cit}R_{cit};
wherein:
R_{cit} is a citrullinated arginine residue;
X is any amino acid; and
X₁ is a non-citrullinated arginine, or any other amino acid.

22. The peptide of claim 16, wherein the peptide is a variant peptide comprising or consisting of any one of the sequences of:
EHSIQFAEMKLR_{cit}PSNFR_{cit}NLEGR_{cit}RKR;
EHSIQFAEMKLR_{cit}PSNFR_{cit}NLEGRR_{cit}KR;
EHSIQFAEMKLR_{cit}PSNFRNLEGR_{cit}R_{cit}KR; or
EHSIQFAEMKLRPSNFR_{cit}NLEGR_{cit}R_{cit}KR; wherein R_{cit} is a citrullinated arginine residue.

23. The peptide of claim 16, wherein the peptide is a variant peptide comprising or consisting of any one of the sequences of:
EHSIQFAEMKLR_{cit}PSNFR_{cit}NLEGR_{cit}RKRA;
EHSIQFAEMKLR_{cit}PSNFR_{cit}NLEGRR_{cit}KRA;
EHSIQFAEMKLR_{cit}PSNFRNLEGR_{cit}R_{cit}KRA; or
EHSIQFAEMKLRPSNFR_{cit}NLEGR_{cit}R_{cit}KRA; wherein R_{cit} is a citrullinated arginine residue.

24. The peptide of claim 16, wherein the peptide is a variant peptide comprising or consisting of any one of the sequences of:
EHSIQFAEMKLR_{cit}PSNFR_{cit}NLEGR_{cit}RKR_{cit};
EHSIQFAEMKLR_{cit}PSNFR_{cit}NLEGRR_{cit}KR_{cit};
EHSIQFAEMKLR_{cit}PSNFRNLEGR_{cit}R_{cit}KR_{cit}; or
EHSIQFAEMKLRPSNFR_{cit}NLEGR_{cit}R_{cit}KR_{cit}; wherein R_{cit} is a citrullinated arginine residue.

25. The peptide of claim 16, wherein the peptide is a variant peptide comprising or consisting of any one of the sequences of:
EHSIQFAEMKLR_{cit}PSNFR_{cit}NLEGR_{cit}RKR_{cit}A;
EHSIQFAEMKLR_{cit}PSNFR_{cit}NLEGRR_{cit}KR_{cit}A;
EHSIQFAEMKLR_{cit}PSNFRNLEGR_{cit}R_{cit}KR_{cit}A; or
EHSIQFAEMKLRPSNFR_{cit}NLEGR_{cit}R_{cit}KR_{cit}A; wherein R_{cit} is a citrullinated arginine residue.

26. The peptide according to claim 2 16 to 25, wherein one or more non-citrullinated amino acid residues are removed or added, such that the spacing between the citrullinated arginine residues is varied.

27. The peptide according to claims 16 to 26, wherein the peptide comprises a N-terminal cysteine and a C-terminal cysteine.

28. The peptide according to claim 16, wherein the peptide is cTNC5 peptide described herein.

29. A biomarker for determining the inflammatory disorder status, of a subject wherein the biomarker comprises:
(i) citrullinated tenascin-C or a fragment thereof which is citrullinated at at least three arginine residues of residue numbers 2187, 2192, 2197, 2198, and 2200; and/or
(ii) autoantibodies with specificity for an epitope of citrullinated tenascin-C or a fragment thereof wherein the epitope comprises at least three citrullinated arginine residues of 2187, 2192, 2197, 2198 and 2200.

30. The biomarker of claim 29, wherein the citrullinated tenascin-C or a fragment thereof is citrullinated at at least three arginine residues of residue numbers 2187, 2192, 2197, and 2198.

31. The biomarker of claim 29, wherein the epitope comprises at least three citrullinated arginine residues of 2187, 2192, 2197, and 2198.

32. The biomarker of claim 29 or 30, wherein the biomarker comprises citrullinated tenascin-C or a fragment thereof which is citrullinated at arginine residues 2187, 2192, 2197 and 2198.

33. The biomarker of any of claims 29 to 32, wherein the biomarker comprises citrullinated tenascin-C or a fragment thereof which is citrullinated at arginine residues 2187, 2192, 2197, 2198 and 2200.

34. The biomarker of claims 29 or 31, wherein the biomarker comprises autoantibodies with specificity for an epitope of citrullinated tenascin-C or a fragment thereof wherein the epitope comprises citrullinated arginine residues 2187, 2192, 2197 and 2198.

35. The biomarker of any of claims 29, 31 or 34, wherein the biomarker comprises autoantibodies with specificity for an epitope of citrullinated tenascin-C or a fragment thereof wherein the epitope comprises citrullinated arginine residues 2187, 2192, 2197, 2198, and 2200.

35. The biomarker of any of claims 29 to 35, wherein the inflammatory disorder is rheumatoid arthritis or pre-rheumatoid arthritis.

36. A method of determining the inflammatory disorder status of a subject comprising detecting the presence or absence, or the level, of a biomarker in a sample from said subject, wherein the biomarker comprises:
(i) citrullinated tenascin-C or a fragment thereof which is citrullinated at at least three arginine residues of residue numbers 2187, 2192, 2197 2198 and 2200; and/or
(ii) detecting the presence or absence, or the level, of autoantibodies with specificity for an epitope of citrullinated tenascin-C or a fragment thereof wherein the epitope comprises at least three citrullinated arginine residues of 2187, 2192, 2197 2198 and 2200.

37. The method according to claim 36, wherein the citrullinated tenascin-C or fragment thereof is citrullinated at at least three arginine residues of residue numbers 2187, 2192, 2197, and 2198.

38. The method according to claim 36, wherein the epitope comprises at least three citrullinated arginine residues of 2187, 2192, 2197, and 2198.

39. The method according to any of claims 36 to 2438 wherein the citrullinated tenascin-C or fragment thereof is citrullinated at arginine residues 2187, 2192, 2197 and 2198, and/or or the epitope comprises citrullinated arginine residues 2187, 2192, 2197 and 2198.

40. The method according to any of claims 36 to 39, wherein the citrullinated tenascin-C or fragment thereof is citrullinated at arginine residues 2187, 2192, 2197, 2198 and 2200, and/or or the epitope comprises citrullinated arginine residues 2187, 2192, 2197, 2198 and 2200.

41. The method according to any of claims 36 to 40, wherein the epitope is on a fragment of cTNC.

42. The method according to any of claims 36 to 41, wherein the method comprises detecting the presence or absence, or the level, of autoantibodies with specificity for cTNC5 described herein.

43. The method of any of claims 36 to 42, wherein the level of the biomarker detected in the sample is compared with one or more reference values.

44. The method of any of claims 36 to 43, wherein the presence of the biomarker in a sample from said subject is sufficient to conclude the subject has an inflammatory disorder.

45. The method of any of claims 36 to 44, wherein the inflammatory disorder is RA.

46. A method of monitoring the progression of an inflammatory disease or monitoring the efficacy of a treatment administered to a subject comprising detecting the level of
(i) citrullinated tenascin-C or a fragment thereof which is citrullinated at at least three arginine residues of residue numbers 2187, 2192, 2197 2198 and 2200; and/or
(ii) autoantibodies with specificity for an epitope of citrullinated tenascin-C or a fragment thereof wherein the epitope comprises at least three citrullinated arginine residues of 2187, 2192, 2197 2198 and 2200;
wherein the detection is in a sample from said subject, and
comparing the levels to normal and/or reference values.

47. The method according to claim 46, wherein the citrullinated tenascin-C or fragment thereof is citrullinated at at least three arginine residues of residue numbers 2187, 2192, 2197, and 2198.

48. The method according to claim 46, wherein the epitope comprises at least three citrullinated arginine residues of 2187, 2192, 2197, and 2198.

49. The method according to claim 46, wherein the citrullinated tenascin-C or fragment thereof is citrullinated at arginine residues 2187, 2192, 2197 and 2198 or the epitope comprises citrullinated arginine residues 2187, 2192, 2197 and 2198.

50. The method according to any of claims 46 to 49, wherein the citrullinated tenascin-C or fragment thereof is citrullinated at arginine residues 2187, 2192, 2197, 2198 and 2200, and/or the epitope comprises citrullinated arginine residues 2187, 2192, 2197, 2198 and 2200.

51. The method of any of claims 46 to 50, wherein the reference values are the initial levels in the subject, or the levels in the subject when they were previously tested, or both.

52. A kit for use in determining the inflammatory disorder status of a subject comprising at least one agent for detecting the presence, or the level, of
(i) citrullinated tenascin-C or a fragment thereof which is citrullinated at at least three arginine residues of residue numbers 2187, 2192, 2197 2198 and 2200; and/or
(ii) autoantibodies with specificity for an epitope of citrullinated tenascin-C or a fragment thereof wherein the epitope comprises at least three citrullinated arginine residues of 2187, 2192, 2197 2198 and 2200;
wherein the detection is in a sample provided by the subject.

53. The kit of claim 52 wherein the agent comprises the biomarker according to any of claims 29-35.

54. The kit according to any of claim 52 to 53, wherein the citrullinated tenascin-C or fragment thereof is citrullinated at at least three arginine residues of residue numbers 2187, 2192, 2197, and 2198.

55. The kit according to any of claim 52 to 54, wherein the epitope comprises at least three citrullinated arginine residues of 2187, 2192, 2197, and 2198.

56. The kit according to claim 52, wherein the citrullinated tenascin-C or fragment thereof is citrullinated at arginine residues 2187, 2192, 2197 and 2198 or the epitope comprises citrullinated arginine residues 2187, 2192, 2197 and 2198.

57. The kit according to any of claim 52 to 56, wherein the citrullinated tenascin-C or fragment thereof is citrullinated at arginine residues 2187, 2192, 2197, 2198 and 2200, and/or the epitope comprises citrullinated arginine residues 2187, 2192, 2197, 2198 and 2200.

58. The kit according to any of claims 52 to 57, wherein the agent comprises a peptide according to any of claims 1 to 13.

59. The kit according to any of claims 52 to 58, wherein the autoantibodies have specificity for cTNC5 described herein.

60. The kit according to any of claims 52 to 59, wherein the kit further comprises a panel of peptides and/or antibodies for detecting a panel of biomarkers for the inflammatory condition.

61. Use of the determination of the presence, or the level, of the biomarker according to any of claims 29 to 35 in a sample obtained from a subject, as a means of assessing the inflammatory disorder status in the subject.

62. Use of:
(i) citrullinated tenascin-C or a fragment thereof which is citrullinated at at least three arginine residues of residue numbers 2187, 2192, 2197 2198 and 2200; and/or
(ii) autoantibodies with specificity for an epitope of citrullinated tenascin-C or a fragment thereof wherein the epitope comprises at least three citrullinated arginine residues of 2187, 2192, 2197 2198 and 2200;
as a biomarker for an inflammatory disorder.

63. The use according to claim 61 or claim 62, wherein the citrullinated tenascin-C or fragment thereof is citrullinated at at least three arginine residues of residues 2187, 2192, 2197 and 2198 or the epitope comprises at least three citrullinated arginine residues at residues 2187, 2192, 2197 and 2198.

64. The use according to claim 61 or claim 62, wherein the citrullinated tenascin-C or fragment thereof is citrullinated at arginine residues 2187, 2192, 2197 and 2198 or the epitope comprises citrullinated arginine residues 2187, 2192, 2197 and 2198.

65. The use according to any of claims 61 or 62, wherein the citrullinated tenascin-C or fragment thereof is citrullinated at arginine residues 2187, 2192, 2197, 2198 and 2200, and/or the epitope comprises citrullinated arginine residues 2187, 2192, 2197, 2198 and 2200.

66. The method, kit, biomarker or use of any preceding claim wherein the inflammatory disorder is selected from the group comprising rheumatoid arthritis (RA), autoimmune conditions, inflammatory bowel diseases (including Crohn's disease and ulcerative colitis), nonhealing wounds, multiple sclerosis, cancer, atherosclerosis, sjogrens disease, diabetes, lupus erythematosus (including systemic lupus erythematosus), asthma, fibrotic diseases (including liver cirrhosis), pulmonary fibrosis, UV damage, psoriasis, psoriatic arthritis, ankylosing spondylitis, myositis and cardiovascular disease.

67. The method, kit, biomarker or use of claim 66 wherein the inflammatory disorder is rheumatoid arthritis.

68. The method, kit, biomarker or use of any of claims 16 to 67 wherein the presence of the biomarker is diagnostic of an inflammatory condition; or prognostic for RA in the presence of synovial inflammation.

69. The method, kit, biomarker or use of any of claims 16 to 68 wherein the presence of the biomarker is prognostic of an inflammatory condition at least 5 years before onset of the condition.

70. The method, kit, biomarker or use of claim 69 wherein the inflammatory condition is rheumatoid arthritis.

71. The method, kit, biomarker or use of any of claims 16 to 70 wherein the sample is blood, serum, plasma, synovial fluid and/or joint tissue derived from the subject.

72. The method, kit, biomarker or use of any of claims 16 to 71 wherein the sample is pre-RA serum.

73. A binding member capable of specifically binding to a peptide according to any of claims 16 to 28, or a biomarker according to any of claims 29 to 35.

74. The binding member according to claim 73, wherein the binding member competes for binding with an autoantibody with specificity for an epitope of citrullinated tenascin-C or a fragment thereof wherein the epitope comprises at least three citrullinated arginine residues of 2187, 2192, 2197 2198 and 2200.

75. The binding member according to claim 73, wherein the binding member competes for binding with an autoantibody with specificity for an epitope of citrullinated tenascin-C or a fragment thereof wherein the epitope comprises at least three citrullinated arginine residues of 2187, 2192, 2197 and 2198.

76. The binding member according to claims 73 or 74, wherein the binding member competes for binding with an autoantibody with specificity for an epitope of citrullinated tenascin-C or a fragment thereof wherein the epitope comprises citrullinated arginine residues 2187, 2192, 2197 and 2198.

77. The binding member according to any of claims 73 to 76, wherein the binding member competes for binding with an autoantibody with specificity for an epitope of citrullinated tenascin-C or a fragment thereof wherein the epitope comprises citrullinated arginine residues 2187, 2192, 2197, 2198 and 2200.

78. The binding member according to any of claims 73 to 77, wherein the binding member is an antibody, antibody fragment or mimetic thereof.

79. The binding member according to any of claims 73 to 78, wherein the binding member has at least 10-fold higher affinity for binding to
(i) a peptide according to any of claims 16 to 28 relative to an equivalent non-citrullinated peptide; or
(ii) a citrullinated tenascin-C, or fragment thereof, biomarker according to any of claims 29 to 35 relative to an equivalent non-citrullinated tenascin-C or fragment thereof.

80. The binding member according to claim 78, wherein the affinity is at least 100-fold higher.

81. Use of the binding member according to any of claims 73 to 80, for the detection of the peptide according to any of claims 1 to 13, or the biomarker according to any of claims 14 to 21.

82. The use according to claim 81, wherein the detection is in a sample from a mammal, or in vivo in a mammal.

83. The use according to claim 81, wherein the mammal is human.

84. A peptide, method, kit, biomarker, binding member or use substantially as described herein, optionally with reference to the accompanying figures.
